(19) United States
(12) Patent Application Publication
(10) Pub. No.: US 2006/0057156 A1
(43) Pub. Date: Mar. 16, 2006

(54) Materials and methods for inductions of immune tolerance

(76) Inventors: Robert Norman Barker, Aberdeen (GB); Neil andrew Marshall, Aberdeen (GB); Mark Adrian Vickers, Aberdeen (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/519,044

(22) PCT Filed: Jun. 24, 2003
(86) PCT No.: PCT/GB03/02739

§ 371 Date: Sep. 2, 2005

(30) Foreign Application Priority Data
Jun. 24, 2002 (GB) 0214528.2

(51) Int. Cl. A61K 48/00; A61K 39/00

(52) U.S. Cl. 424/192.1; 514/44

(57) ABSTRACT

The invention provides methods of inducing immune tolerance to target antigens in individuals seropositive for certain infectious agents by administration of epitopes derived from those infectious agents. Epitopes derived from viruses which carry homologues of interleukin 10 (IL-10) in their genome, such as Epstein Barr Virus and cytomegalovirus, are particularly suitable for these purposes. Particularly preferred is the use of the EBV LMP1 protein and epitopes thereof.

### FIELD OF THE INVENTION

The present invention relates to induction of immune tolerance, and in particular to the use of epitopes from infectious agents to induce immune tolerance to other antigens in individuals seropositive for those infectious agents.

### BACKGROUND TO THE INVENTION

A wide variety of strategies are employed by viruses to evade immune mediated clearance, which can be considered to belong to one of three major mechanisms: escape, resistance and counterattack (Xu et al., 2001). Viruses escape immune recognition by disruption of antigen presentation pathways (Lorenzo et al., 2001) and epitope mutation (Erickson et al., 2001). Resistance is mediated by inhibiting apoptosis of virally infected cells. Counterattack comprises the killing of effector T cells (Mueller et al., 2001). Epstein-Barr virus (EBV) has been shown to avoid detection and clearance by such mechanisms. For example, escape of detection in Burkitt's lymphoma, where the cells express very low levels of MHC class I and adhesion molecules (Gregory et al., 1988), and Epstein-Barr Nuclear Antigen 1 escapes MHC class I presentation via its NH₂ terminal Gly/Ala repeat domain (Levitskaya et al., 1995, 1997). EBV also evades immune responses by resistance to apoptosis through its Bcl-2 homologue, BHRF-1 (Xu et al., 2001) and expression of the anti-apoptotic protein A20 induced by LMP1 (Fries et al., 1996). However, although EBV has been shown to avoid detection and clearance by these, and other mechanisms, it is still not clear how the virus maintains latent infection so successfully.

EBV is a human γ-herpes virus carried as a latent infection by more than 90% of adults, replicating in B-cells and nasopharyngeal epithelial cells (Kieff, 1996). The acute infection is controlled by a cytotoxic response predominantly against EBV Nuclear Antigens 3A, 3B and 3C (Kieff, 1996), but, in all cases, the virus enters a latent state in B-cells (Kieff, 1996). LMP1 is part of a restricted panel of genes expressed during latency, and in several EBV-associated malignancies including Hodgkin's disease and nasopharyngeal carcinoma (Horikawa et al., 2000; Pallesen et al., 1991). The protein acts as a constitutively activated tumor necrosis factor receptor, transforming cells through activation of molecules including nuclear factor kappa B and the anti-apoptotic protein A20 (Eliopoulos et al., 1996, 1997; Huen et al., 1995; Mosialos et al., 1995; Young et al., 1998).

The role of different CD4⁺ T helper (Th) cell subsets in regulating the nature and efficacy of immune responses is increasingly recognized (Christensen et al., 2001; Groux et al., 1997; Levings and Roncarolo, 2000; Roncarolo et al., 2000, 2001; Shevach et al., 1998; Stephens and Mason, 2000; Thomsen et al., 2001). Initially, attention focused on mutual antagonism between Th1 and Th2 cells, which produce γ-interferon (γ-IFN) and IL-4 respectively (Mossman and Coffman, 1989), but further, T regulatory (Tr) cell subpopulations with important roles in immunoregulation and tolerance have now been defined (Groux et al., 1997; Levings and Roncarolo, 2000; Roncarolo et al., 2000, 2001; Shevach et al., 1998; Stephens and Mason, 2000). In particular, production of the Tr1 cytokine IL-10 can protect rodents against a number of immune-mediated diseases (Groux et al., 1997; Levings and Roncarolo, 2000), whilst Th3 cell secretion of transforming growth factor-β prevents spontaneous autoimmunity (Gorelik and Flavell, 2000) and mediates some forms of oral tolerance (Weiner, 1997). Regulatory subpopulations characterized by CD25 expression have also been isolated from rodents (Seddon, and Mason, 2000; Shevach, 2000), and more recently from human peripheral blood (Jonuleit, et al., 2001; Levings, et al., 2001), but in most reports the suppressive effects of these cells are non-specific and not dependent on cytokine production. The importance of Tr cells in controlling immune-mediated disease raises the prospect that viruses may exploit such regulation as a fourth major mechanism to evade immune clearance. Given that cells latently infected with EBV express LMP1, the question arises as to why this antigen fails to elicit protective cytotoxic immunity (Chapman et al., 2001; Khanna et al., 1998); cytotoxic T cells specific for LMP1 are notable for their absence from infected individuals (Chapman et al., 2001). Dukers et al. (2000) have recently suggested that LMP1 contains peptide motifs which can exert direct immunosuppressive effects on peripheral blood mononuclear cells.

### DISCLOSURE OF THE INVENTION

The present inventors have found that certain infectious agents encode antigens comprising tolerogenic peptide sequences. By a “tolerogenic” peptide sequence is meant a sequence which, when administered to cells of the immune system, along with a target antigen, tolerises the cells to that target antigen.

Exposure to the tolerogenic sequence and the target antigen inhibits the capacity of the cells to mount an immune response to that target antigen on a subsequent challenge, regardless of whether or not the tolerogenic sequence is present for that subsequent challenge. However, although tolerised to the target antigen, populations of cells so treated retain their capacity to mount a response to other antigens in the absence of the tolerogenic sequence.

The types of immune response which can be inhibited in this way include “defensive” immune responses against foreign antigens, including those administered therapeutically, as well as “pathogenic” immune responses as seen in autoimmune and allergenic diseases. These responses are often characterised by lymphocyte proliferation, expression of cytokines such as IL-4 or gamma-IFN, and induction of antibody response.

The cells to be tolerised will be from an individual who has previously been infected with the infectious agent from which the tolerogenic peptide is derived.

Thus these tolerogenic sequences can induce antigen-specific tolerance of mononuclear leukocytes to target antigens. This activity therefore contrasts with the non-specific immunosuppressive effects attributed to some virus-derived peptides, e.g. from retroviral envelope proteins (Haraguchi et al., 1995) and EBV LMP1 protein (Dukers et al., 2000).

The present inventors have shown that it is possible to identify such sequences by testing their ability to induce expression of IL-10 in cells from a donor seropositive for the relevant infectious agent.

Accordingly, in a first aspect, the present invention provides a method for assessing the tolerogenicity of a test peptide sequence from an infectious agent, comprising the steps of:
(i) contacting a cell population with said test peptide sequence,
(ii) determining whether IL-10 expression in said cell population is increased, and optionally
(iii) correlating the result of step (ii) with the tolerogenicity of the sequence,
   wherein said cell population comprises mononuclear leukocytes from a donor previously infected by said infectious agent.

The term “mononuclear leukocytes” as used herein embraces T lymphocytes (including CD4⁺ and CD8⁺ T lymphocytes), B lymphocytes, natural killer (NK) cells, mononuclear phagocytes (monocytes and macrophages) and dendritic cells. Thus the cell population comprises one or more of these types of cells.

Preferably, the cell population comprises at least T lymphocytes, preferably CD4⁺ lymphocytes, or at least one type of antigen presenting cell (APC). More preferably, the cell population comprises at least T lymphocytes, preferably CD4⁺ lymphocytes, and at least one type of antigen presenting cell. An antigen presenting cell is any cell capable of presenting an antigen to a T lymphocyte in the context of an MHC class II molecule. Thus B lymphocytes, natural killer (NK) cells, mononuclear phagocytes (monocytes and macrophages) and dendritic cells are all considered to be APCs. However, the majority of nucleated cells are capable of acting as APCs under the appropriate conditions, e.g. when exposed to pro-inflammatory cytokines, and so the cell population may further comprise APCs which would not normally be regarded as mononuclear leukocytes.

The cell population comprises mononuclear leukocytes derived from a donor previously infected by the relevant infectious agent. Preferably it can be demonstrated by an appropriate assay that the donor has previously raised an immune response against the infectious agent; for example, the donor may be seropositive for the infectious agent, i.e. have circulating antibodies specific for the infectious agent. Under some circumstances the donor may not have circulating antibodies specific for the infectious agent, for example where insufficient time has elapsed since infection for detectable levels of antibodies to be raised, or where a substantial time has elapsed since infection and antibody levels have fallen below the threshold of detectability. However, the term “seropositive” will be used throughout this specification to refer to any individual previously infected by the relevant infectious agent, regardless of actual serological status, and the term “seronegative” should be construed accordingly, i.e as referring to an individual not previously infected by the infectious agent.

The method may further comprise the steps of:
(i)(a) contacting a similar cell population from a donor not previously infected by said infectious agent with said test peptide sequence, and
(ii)(a) determining whether IL-10 expression in said cell population is increased, and optionally
(ii)(b) comparing the results from step (ii) with the results from step (ii)(a).

In step (iii), the individual results, or any combination of the results, from any of steps (ii), (ii)(a) and (ii)(b) may be correlated with the tolerogenicity of the sequence. In general it is considered that the greater the level of IL-10 expression induced in the seropositive population by the test peptide, the more likely it is that the test peptide will be tolerogenic.

Whether or not IL-10 expression is increased may be determined by any appropriate method. Suitable methods include specific detection of IL-10 protein, e.g. by ELISA (Deveraux et al., 2000), flow cytometry (Kreft et al., 1992), non-competitive flow immunoassay (Kjellstrom et al., 2000), immunofluorescence (Scheffold et al., 2000) or immunoblot; by detection of IL-10 mRNA, e.g. by RT-PCR (Blaschke et al., 2000; Demay et al., 1996), or Northern blot; or by bioassay for IL-10 activity (Schlaak et al., 1994).

The present invention further provides a method for assessing the tolerogenicity of a test peptide sequence from an infectious agent towards a target antigen, comprising the steps of:
(i) contacting a cell population with (a) said test peptide sequence and (b) a target antigen, to make a test composition, and
(ii) re-contacting the cell population from said test composition with said target antigen.
   wherein said cell population comprises mononuclear leukocytes from a donor previously infected by said infectious agent. Preferably, the cell population comprises at least one type of APC, which may or may not be a mononuclear leukocyte, as set out above.

In general the cell population will not be re-contacted with the test peptide in step (ii).

The method may further comprise the steps of:
(iii) assessing the response of said cell population to said target antigen, and optionally
(iv) correlating the result of step (iii) with the tolerogenicity of the test peptide sequence.

The response of the cell population to the second challenge with the target antigen may be assessed by any method that enables a tolerised population to be distinguished from a non-tolerised population. For example, a response of a non-tolerised population to a foreign antigen would be expected to include one or more of e.g. cell proliferation (typically lymphocyte proliferation), and expression of one or more cytokines (other than IL-10) such as IL-4, IL-2, IL-12 and gamma-IFN. Thus step (iii) may comprise the assessment of any one of these markers, or of any other suitable marker.

The method may be performed in vivo or in vitro. Preferably the method is performed in vitro, e.g. in culture. However the methods may be performed in any suitable model in vivo.

The purpose of re-contacting the cells with the target antigen in step (ii) is to confirm that the cells have been tolerised to the target antigen by the initial contact of step (i).

Therefore it is desirable that for step (ii), the test composition does not still contain appreciable amounts of the test peptide sequence, or of tolerogenic or immunosuppressive factors produced by the cells themselves, which might interfere with any reaction stimulated by the target antigen in step (ii). Therefore, the method may include the step of allowing the cells to rest between steps (i) and (ii), so that the activity of test peptide in the test composition is reduced, the cells are not still expressing tolerogenic factors which would interfere with any reaction in step (ii), and the activity of residual tolerogenic factors produced by the cells during or in response to the initial tolerogenic challenge is reduced. IL-10 activity is used herein as a marker for tolerogenic factors generated by the PBMCs in step (i).

When performed in vitro, the method may additionally or alternatively comprise the step of washing the cells prior to step (ii). Washing may be performed in conventional fashion. Typically, the cells will be rested after washing. Fresh antigen presenting cells may be added before recontacting the cells with the target antigen in step (ii).

Without wishing to be bound by any particular theory, it is believed that IL-10 may play an effector role in inducing tolerance, so reduction of IL-10 activity may also be achieved by specific neutralisation, e.g. addition of a neutralising factor to the cells, such as a neutralising anti-IL-10 antibody.

The method may further comprise the step of contacting the cell population with a confirmatory antigen unrelated to the test sequence or the target antigen, to confirm that the cells retain their general reactive capability, even though their reactivity to the target antigen has been modified.

Any suitable antigen may be used as the target antigen or confirmatory antigen. These antigens may be primary antigens or recall antigens; that is to say, the cells in the assay may or may not have been exposed to them before. A typical primary antigen for assay use is KLH (keyhole limpet haemocyanin), while for donors previously immunised with Bacille Calmette-Guérin (BCG), purified protein derivative (PPD) from Mycobacterium tuberculosis is a suitable recall antigen. T cell mitogens such as Concanavalin A, which are generally regarded as relatively non-specific in their activation of T cells, can also be used as target or confirmatory antigens within the meaning of the present invention. It has been found that PBMCs can be rendered unresponsive to ConA, PPD and other antigens or stimuli by the techniques described herein, but still retain their ability to respond to other antigens.

A test peptide sequence which is capable of inducing IL-10 expression and/or antigen-specific tolerance in seropositive cells as described above may be regarded as a “tolerogenic peptide sequence”.

A tolerogenic peptide sequence may therefore be used to modulate an immune response, either in vivo or in vitro, by administration to suitable seropositive mononuclear leukocytes along with a target antigen. This technique has a number of applications. For example, it may be used prophylactically, to prevent subsequent development of an inflammatory response to the target antigen, or to inhibit a pre-existing immune reaction to the target antigen.

Accordingly, in a further aspect, the present invention provides a method of tolerising a cell population to a target antigen, comprising contacting said cell population with
(a) a tolerogenic peptide sequence from an infectious agent, and
(b) the target antigen,
   wherein said cell population comprises mononuclear leukocytes from a donor seropositive for said infectious agent.

The cell population may be contacted with the tolerogenic peptide sequence and/or the target antigen directly. Alternatively, the cell population may be contacted with the tolerogenic peptide sequence and/or the target antigen indirectly, e.g. via APCs which would not normally be regarded as mononuclear leukocytes, as described above. Thus a population of APCs may be contacted with the tolerogenic peptide sequence and/or the target antigen, and the cell population subsequently contacted with the population of APCS.

The tolerogenic peptide and target antigen may be administered to the cell population, or to the population of APCs, either together or separately, and in any order. Thus it is not intended that the tolerogenic peptide sequence and target antigen must necessarily be administered simultaneously.

Any or all of the steps described may be performed in vitro, in vivo, or ex vivo.

Thus all the steps described may be performed in vitro, e.g. in culture.

In some embodiments, a tolerogenic peptide sequence and a target antigen may be administered directly to a test subject or a subject to be treated, e.g. an individual who has previously been infected by the relevant infectious agent. Thus the invention provides a method of treatment of a disease or condition mediated by an immune response against a target antigen, comprising administering a tolerogenic peptide sequence to an individual suffering from said condition or disease. The target antigen may also be administered, either with the tolerogenic peptide sequence or separately.

In alternative embodiments, a tolerogenic peptide sequence and a target antigen may be administered in vitro to a cell population comprising mononuclear leukocytes from such an individual. These cells may then be introduced into a test subject, or a subject to be treated, e.g. the subject from whom they were originally derived.

In alternative embodiments, a tolerogenic peptide sequence and a target antigen may be administered in vitro to a population of APCs. The population of APCs may then be contacted in vitro with a cell population comprising mononuclear leukocytes from an infected individual. That cell population, or a subset thereof e.g. some or all of the mononuclear leukocytes, may then be introduced into a test subject, or a subject to be treated, e.g. the subject from whom they were originally derived.

Alternatively, the population of APCs may be administered to a test subject, or a subject to be treated, e.g. the subject from whom they were originally derived. In this case contact between the cell population and the tolerogenic peptide sequence and target antigen takes place in vivo, via the APCs.

Thus cells or tissues may be removed from a donor individual or individual to be treated, treated with the tolerogenic peptide sequence and a target antigen, and reintroduced to the donor. Suitable cells or tissues include particular type of antigen presenting cells, heterogeneous populations of cells, e.g. peripheral blood lymphocytes or subsets thereof, lymph nodes, etc.

Preferably, the cell population comprises at least T lymphocytes, preferably CD4⁺ T lymphocytes. More preferably, the cell population comprises at least T lymphocytes, preferably CD4⁺ T lymphocytes, and at least one type of APC. From the above description it can be seen that the cell population to be tolerised, may in some embodiments be considered to comprise cells in situ in a test subject or subject to be treated.

The test subject, or subject to be treated will typically be a mammal, and may be a human. In some embodiments, a test subject may be a non-human mammal e.g. a rodent, rabbit, etc. and will typically be seropositive for the infectious agent.

Certain infectious agents do not have animal models that are easy to manipulate. For example, the human pathogen EBV has no animal model. Therefore the test subject may be a non-human mammal with a severe combined immunodeficiency, comprising lymphocytes from a donor of the appropriate species seropositive for the infectious agent. By “severe combined immunodeficiency” is meant a defect in lymphocyte maturation, so that the affected animal has low or undetectable levels of mature T and/or B lymphocytes. The mammal may be a rodent, for example a mouse or rat, such as the SCID mouse. In preferred embodiments the non-human mammal with the severe combined immunodeficiency is reconstituted with human lymphocytes seropositive for EBV, e.g. from a seropositive donor. Suitable techniques are described in Mosier et al. (1988), McCune et al. (1988), Kamel-Reid et al. (1988), and Rowe et al. (1991). Similar techniques may be applied to create animal models of other conditions.

In any of the embodiments of the present invention, the target antigen may be a suitable test antigen as described above, or any antigen to which an inappropriate or undesirable immune response occurs or is likely to occur. Thus the target antigen may be one implicated in a disease state, e.g. a self antigen implicated in an autoimmune condition, such as rheumatoid arthritis, or an allergic state such as hayfever. The target antigen maybe a protein, polypeptide or peptide, including an epitope of a protein, or any other suitable entity capable of provoking an immune reaction, such as polysaccharides, lipids, macromolecular complexes, cells, etc.

Examples of auto-immune diseases in which specific antigens have been identified as potentially pathogenically significant include multiple sclerosis (myelin basic protein), insulin-dependent diabetes mellitus (glutamic acid decarboxylase), insulin-resistant diabetes mellitus (insulin receptor), coeliac disease (gliadin), bullous pemphigoid (collagen type XVII), auto-immune haemolytic anaemia (Rh protein), auto-immune thrombocytopenia (GpIIb/IIIa), myaesthenia gravis (acetylcholine receptor), Graves' disease (thyroid-stimulating hormone receptor), glomerulonephritis, such as Goodpasture's disease (alpha3(IV)NC1 collagen), and pernicious anaemia (intrinsic factor). Thus these antigens, or particular fragments or epitopes thereof may be suitable target antigens. The target antigen may be an exogenous antigen which stimulates a response which also causes damage to host tissues. For example, acute rheumatic fever is caused by an antibody response to a Streptococcal antigen which cross-reacts with a cardiac muscle cell antigen. The target antigen may be one which provokes an atopic or allergic response, e.g. pollen (implicated in hayfever, e.g. Timothy Grass pollen), house dust mites (asthma), cosmetics, allergens administered via insect bites, nut allergens, or therapeutic products such as factor VIII, factor IX, blood group antigens, or monoclonal antibodies.

The methods of the present invention may be used to suppress responses to allogeneic or xenogeneic cells or tissues, including primary and secondary mixed lymphocyte reactions, graft rejection, and graft versus host disease. Thus a subject intended to receive a cellular transplant may be tolerised to antigens expressed by those cells. Alternatively, the transplant may be given in conjunction with tolerogenic peptide sequences as described herein, or nucleic acid encoding such peptide sequences, in order to tolerise the recipient to those cells. In preferred embodiments, some or all of the cells to be transplanted may be engineered to express tolerogenic peptides. Thus a cell to be transplanted may contain nucleic acid encoding a tolerogenic peptide sequence according to the present invention such that the cell is capable of expressing the tolerogenic peptide sequence. The optimum methodology will depend on the identity of the cells to be engineered. Antigen presenting cells, e.g. dendritic cells, etc., may be engineered to express the tolerogenic peptide sequence in such a manner that it is processed and presented in the context of the cells' own MHC II molecules. Other cell types may be engineered so that they secrete the expressed sequence, in order that it can be presented by neighbouring APCs.

In all of the aspects described herein, the infectious agent, from which the test or tolerogenic peptide sequence is derived, may be a virus. In preferred embodiments, the virus is a herpesvirus encoding a viral IL-10 homologue, preferably EBV.

The test or tolerogenic peptide sequence may be derived from an EBV protein, preferably EBV LMP1 protein or LMP2 protein. Thus the methods of the present invention extend to the use of LMP1 protein, LMP2 protein, or a portion or fragment thereof comprising a tolerogenic peptide sequence.

The test or tolerogenic peptide sequence may comprise one or more of the sequences p1 to p75, or p1′ to p96′. If desired, more than one test or tolerogenic peptide sequence may be administered, either simultaneously or sequentially.

The present invention also provides a method of treating a disease mediated by an immune response against a target antigen, comprising the steps of administering (a) a tolerogenic peptide sequence from an infectious agent, and (b) the target antigen, to an individual seropositive for said infectious agent.

Nucleic acids encoding test or tolerogenic peptides, and/or target antigens, may be useful in all the methods of the present invention. As an alternative to administration of a peptide to cells, a nucleic acid encoding that peptide and capable of supporting its expression may be used instead. For example, DNA vaccination techniques are well known to the skilled person, as reviewed in Mor and Eliza (2001); Smith (2000); Schleef et al. (2000) and Apostolopoulos and Plebanski (2000). Thus where administration of a peptide sequence is referred to in any of the methods herein described, administration of a nucleic acid sequence encoding that peptide sequence is also envisaged. Thus contacting a cell population or population of antigen presenting cells with a peptide sequence is considered to encompass contacting the relevant cells with an appropriate nucleic acid.

Thus for example, the present invention further provides a method of tolerising a cell population to a target antigen, comprising contacting said cell population with
(a) a nucleic acid encoding said test peptide sequence, such that said test peptide sequence is expressed in said cell population, and
(b) the target antigen,
   wherein said cell population comprises mononuclear leukocytes from a donor seropositive for said infectious agent.

Where the target antigen is a protein, polypeptide or peptide, a nucleic acid encoding the target antigen may be administered, so that the target antigen is expressed in said cell population. However this should not be taken to imply that the target antigen need necessarily be a protein, polypeptide or peptide.

Use of nucleic acids in this way is considered to be applicable, mutatis mutandis, to any corresponding embodiment of the present invention in which administration of a peptide sequence is referred to. When target antigens are protein or peptide, nucleic acids having appropriate coding sequences may likewise be administered instead. In related embodiments, cells may be contacted with peptides by contact with cells engineered to express the relevant peptides and either secrete them or present them in the context of MHC molecules.

The present invention further provides a pharmaceutical composition comprising a tolerogenic peptide sequence from an infectious agent and a target antigen, in admixture with a pharmaceutically acceptable carrier.

In preferred embodiments, the tolerogenic peptide sequence is derived from EBV, e.g. LMP1 or LMP2 as described above. Thus the composition may comprise EBV LMP1 protein, LMP2 protein, or a portion or fragment of either comprising a tolerogenic peptide sequence.

In preferred embodiments the tolerogenic peptide sequence may comprise one or more of the LMP1 peptide sequences P1 to P75, and/or one or more of the LMP2 peptide sequences P1′ to P96′ described herein.

The present invention further provides EBV LMP1 and LMP2 proteins, and portions or fragments of either, for example, the peptide sequences P1 to P75, or P1′ to P96′ comprising a tolerogenic peptide sequence, for use in a method of medical treatment.

The present invention further provides EBV LMP1 and LMP2 proteins, and portions or fragments thereof, for example, the peptide sequences P1 to P75, or P1′ to P96′ comprising a tolerogenic peptide sequence, for use in the treatment of a condition mediated by an immune response directed against a target antigen.

The present invention further provides EBV LMP1 and LMP2 proteins, and portions or fragments thereof, for example, the LMP1 peptide sequences P1 to P75, and the LMP2 peptide sequences P1′ to P96′ comprising a tolerogenic peptide sequence, in the preparation of a medicament for the treatment of a condition mediated by an immune response directed against a target antigen. The medicament may further comprise the target antigen. The medicament will typically be formulated for administration to an individual previously infected by EBV.

In these and other aspects of the present invention, preferred peptides include P2, P4, P5, P6, P7, P8, P9, P10, P12, P13, P14, P15, P16, P17, P18, P20, P22, P23, P24, P25, P26, P27, P29, P30, P32, P34, P35, P39, P68, P71, P72. Particularly preferred peptides include P2, P4, P7, P14, P15, P18, P20, P22, P23, P24, and P32.

The condition may be, for example, type I diabetes mellitus, coeliac disease, multiple sclerosis, rheumatoid arthritis, systemic lupus erythematosus, myaesthenia gravis, autoimmune haemolytic anaemia and thrombocytopenia, an atopic response e.g. hay fever or asthma, or other allergy, e.g. to an allergen such as a pharmaceutical product or nut allergens, or an alloimmune response, e.g. graft rejection, graft versus host disease, or a response to therapeutic products such as factor VIII, or monoclonal antibody therapy. The target antigens described above may be useful for treatment of these conditions.

The compositions and medicaments described herein may comprise nucleic acids encoding tolerogenic peptides and/or target antigens, as appropriate.

Also provided is a pharmaceutical composition comprising a cell for transplantation to a recipient, in admixture with a pharmaceutically acceptable carrier, said cell comprising nucleic acid encoding a tolerogenic peptide according to the present invention, such that said tolerogenic peptide sequence can be expressed by said cell.

The nucleic acid preferably encodes an EBV protein, e.g. LMP1 or LMP2, or a fragment thereof comprising a tolerogenic peptide sequence.

The tolerogenic peptide sequence and the target antigen may be administered together or separately. In preferred embodiments, they are administered together. They may be provided as an admixture of separate components, as a complex, or covalently associated. Where the target antigen is a protein, the tolerogenic peptide sequence and target antigen may be provided as a fusion protein. Use of fusion proteins in this manner is applicable to all aspects of the invention.

In any of the above-described aspects of the invention, the cell population to be tolerised may comprise mononuclear leukocytes from any suitable species. In preferred embodiments the mononuclear leukocytes are mammalian, e.g. from livestock animals such as horses, cattle, etc., from domestic animals, such as dogs, cats, etc., or from humans. Likewise, individuals to be treated by the methods of the present invention are preferably mammals, e.g. livestock animals such as horses, cattle, etc., domestic animals, such as dogs, cats, etc., and humans.

The term “peptide sequence” as used herein, whether a test or tolerogenic peptide sequence, should not be taken to refer solely to a free peptide consisting essentially or exclusively of that sequence, although this is encompassed by the present invention. Without wishing to be bound by any particular theory, it is believed that the methods of the present invention are effective as long as the relevant sequence can be presented to T cells by antigen presenting cells within the population. Thus it is believed that the test or tolerogenic peptide sequence may constitute a T cell epitope, in that it is capable of being presented to T cells in the context of MHC molecules. Therefore the test or tolerogenic peptide sequence is preferably at least 6 amino acids in length, more preferably at least 8 amino acids in length.

Preferably the test or tolerogenic peptide sequence is capable of acting as an MHC class II-restricted T cell epitope. The chance that a peptide will be capable of acting as a T cell epitope can be determined by assessing its ability to bind to the antigen binding groove of MHC II molecules. Peptide motifs which bind particular MHC alleles are known, and computer programs are available which can identify such motifs within protein sequences (Sturniolo et al. (1999); Singh and Raghava (2001)).

The skilled person will be aware that any T cell that responds to a given peptide can also respond in a similar way to other peptides containing substitutions in residues that are not critical for MHC binding or T cell receptor recognition, and even to certain peptides that are substituted in critical residues. Such immunological cross reactivity of peptides can be demonstrated by showing that a particular T cell is capable of responding to more than one peptide. Such experiments may be performed using T cell clones. Techniques for cloning T cells are well known in the art. Without wishing to be bound by any particular theory, T cells of Tr1 phenotype may be implicated in the mechanism underlying the methods described herein. Such T cells do not proliferate significantly in response to stimulation, and suppress proliferation of other cells, and so can be difficult to clone. However, suitable techniques are known—see e.g. MacDonald et al. (2002).

Tolerogenic peptides derived from infectious agents described herein, or identified using the methods herein, may be used to screen for immunologically cross reactive peptides which exert similar tolerogenic effects by stimulating a similar or overlapping T cell population. Such cross reactive peptides may be considered ‘mimetics’ of the infectious agent-derived tolerogenic peptides described herein. Thus the present invention provides a method for assessing the tolerogenicity of a test peptide sequence, comprising the steps of:
(i) contacting a first cell population with said test peptide sequence,
(ii) contacting a second cell population with a control peptide sequence
(iii) determining whether IL-10 expression in each said cell population is increased, and optionally
(iv) correlating the result of step (iii) with the tolerogenicity of the test peptide sequence,
   wherein each said cell population comprises mononuclear leukocytes from a donor previously infected by an infectious agent, and said control peptide sequence is derived from said infectious agent. Thus typically, the control peptide sequence will have been previously shown to induce IL-10 expression in a cell population comprising mononuclear leukocytes from a donor previously infected by said infectious agent.

Preferably, the first and second cell populations are derived from the same donor individual. In preferred embodiments the first and second cell populations comprise T cell clones, preferably Tr1 T cell clones, shown to respond to the control peptide when appropriately presented by APCs.

The control peptide may comprise one or more of peptides P1 to P75 and/or P1′ to P96′ described herein.

The skilled person will also be aware that, because of the polymorphic nature of the MHC, most peptides will not be capable of binding to all MHC molecules. Thus compositions for use in the present invention may be tailored to a specific individual, by selecting peptides likely to bind to their MHC. Alternatively, compositions may be designed to have a broader spectrum of activity, being applicable to a wider range of the population. This may be achieved by incorporating peptides capable of binding more than one MHC allele, and/or incorporating more than one test or tolerogenic peptide, each having different MHC specificity. These peptides may be provided in any appropriate form, e.g. as mixtures of separate peptides or as fusion proteins.

Therefore the test or tolerogenic peptide sequence may be administered as part of a longer peptide, polypeptide or protein. For example, the sequence may be used in the context of the whole or part of the full length native protein. The peptide, polypeptide or protein may be administered in any appropriate form, e.g. in native or denatured conformation.

It will be appreciated that any peptide, polypeptide or protein may comprise more than one tolerogenic peptide sequence within the meaning of the present invention. For example, the EBV LMP1 protein is believed to contain numerous individual peptide sequences capable of inducing tolerance to a target antigen in EBV-seropositive PBMCs, as described more fully in the Examples below. Furthermore, a peptide, polypeptide or protein comprising one or more tolerogenic epitopes may be utilised in admixture with target antigen, or may, for example, be provided covalently coupled with a target antigen, either by chemical linkage, or, where the target antigen is a protein, as a fusion protein.

Peptides, polypeptides or proteins, including fusion proteins, for use in the methods or compositions of the present invention may be generated by any appropriate method, including chemical synthesis and recombinant expression.

The present invention further provides individual peptides having any one of the sequences P1 to P75 and P1′ to P96′ described herein. Preferred peptides have the sequences of P2, P4, P5, P6, P7, P8, P9, P10, P12, P13, P14, P15, P16, P17, P18, P20, P22, P23, P24, P25, P26, P27, P29, P30, P32, P34, P35, P39, P68, P71, P72. Particularly preferred peptides have sequences of P2, P4, P7, P14, P15, P18, P20, P22, P23, P24, and P32.

Thus in a further aspect, the present invention provides isolated nucleic acid molecules encoding the test and tolerogenic sequences of the present invention. The open reading frame may be contiguous with an open reading frame encoding a desired target antigen, in order to encode a fusion protein as described above.

In further aspects, the present invention provides an expression vector comprising the above tolerogenic sequence-encoding nucleic acid, operably linked to control sequences to direct its expression, as well as host cells transformed with the vectors. The present invention also includes a method of producing peptides of the preceding aspect, comprising culturing the host cells and isolating the tolerogenic peptides thus produced.

In order to obtain expression of nucleic acids encoding test, tolerogenic or target antigen sequences, the sequences can be incorporated into a vector having control sequences operably linked to the encoding nucleic acid to control its expression. The vectors may include other sequences such as promoters or enhancers to drive the expression of the inserted nucleic acid, nucleic acid sequences so that the tolerogenic sequence peptide is produced as a fusion, e.g. with one or more other such tolerogenic sequences, or with one or more target antigens, and/or nucleic acid encoding secretion signals so that the peptide produced in the host cell is secreted from the cell. Peptides/polypeptides/proteins can then be obtained by transforming the vectors into host cells in which the vector is functional, culturing the host cells so that the peptide is produced and recovering the peptide from the host cells or the surrounding medium. Prokaryotic and eukaryotic cells are used for this purpose in the art, including strains of E. coli, yeast, and eukaryotic cells such as COS or CHO cells.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, “Molecular Cloning: a Laboratory Manual”: 2nd edition, Sambrook et al., 1989, Cold Spring Harbor Laboratory Press.

Cells and techniques may be selected such as to permit or enhance the folding and\or formation of disulphide bridges (see e.g. “Protein Folding” by R. Hermann, Pub. 1993, European Patent Office, The Hague, Netherlands, ISBN 90-9006173-8).

Peptides may be synthesized by any suitable method, such as by exclusively solid-phase techniques, by partial solid-phase techniques, by fragment condensation or by classical solution couplings. In conventional solution phase peptide synthesis, the peptide chain can be prepared by a series of coupling reactions in which the constituent amino acids are added to the growing peptide chain in the desired sequence.

Briefly, N-alpha-protected amino acid anhydrides are prepared in crystallized form or prepared freshly in solution and used for successive amino acid addition at the N-terminus. At each residue addition, the growing peptide (on a solid support) is acid treated to remove the N-alpha-protective group, washed several times to remove residual acid and to promote accessibility of the peptide terminus to the reaction medium. The peptide is then reacted with an activated N-protected amino acid symmetrical anhydride, and the solid support is washed. At each residue-addition step, the amino acid addition reaction may be repeated for a total of two or three separate addition reactions, to increase the percent of growing peptide molecules which are reacted. Typically, 1-2 reaction cycles are used for the first twelve residue additions, and 2-3 reaction cycles for the remaining residues.

The use of various N-protecting groups, various coupling reagents, e.g., dicyclohexylcarbodiimide or carbonyldiimidazole, various active esters, e.g., esters of N-hydroxyphthalimide or N-hydroxysuccinimide, and the various cleavage reagents, to carry out reaction in solution, with subsequent isolation and purification of intermediates, is well known classical peptide methodology. Classical solution synthesis is described in detail in the treatise “Methoden der Organischen Chemie (Houben-Weyl): Synthese von Peptiden”, E. Wunsch (editor) (1974) Georg Thieme Verlag, Stuttgart, W. Ger. Techniques of exclusively solid-phase synthesis are set forth in the textbook “Solid-Phase Peptide Synthesis”, Stewart & Young, Pierce Chemical Co., Rockford, Ill., 1984, and are exemplified by the disclosure of U.S. Pat. No. 4,105,603. The fragment condensation method of synthesis is exemplified in U.S. Pat. No. 3,972,859. Other available syntheses are exemplified by U.S. Pat. Nos. 3,842,067 and 3,862,925. Peptides are preferably prepared using the Merrifield solid phase synthesis, although other equivalent chemical syntheses known in the art can also be used as previously mentioned. Such solid-phase synthesis is commenced from the C-terminus of the peptide by coupling a protected alpha-amino acid to a suitable resin. Such a starting material can be prepared by attaching an alpha-amino-protected amino acid by an ester linkage to a chloromethylated resin or a hydroxymethyl resin, or by an amide bond to a benzhydrylamine (BHA) resin or paramethylbenzhydrylamine (MBHA) resin. The preparation of the hydroxymethyl resin is described by Bodansky et al., Chem. Ind. (London) 38, 1597-98 (1966). Chloromethylated resins are commercially available from Bio Rad Laboratories, Richmond, Calif. and from Lab. Systems, Inc. The preparation of such a resin is described by Stewart et al., “Solid Phase Peptide Synthesis”, supra.

The C-terminal amino acid, protected by Boc and by a side-chain protecting group, if appropriate, can be first coupled to a chloromethylated resin according to the procedure set forth in Chemistry Letters, K. Horiki et al. 165-168 (1978), using KF in DMF at about 60° C. for 24 hours with stirring, when a peptide having free acid at the C-terminus is to be synthesized.

Conditions for removal of specific alpha-amino protecting groups may be used as described in Schroder & Lubke, “The Peptides”, 1 pp 72-75, Academic Press (1965).

Activating reagents and their use in peptide coupling are described by Schroder & Lubke supra, in Chapter III and by Kapoor, J. Phar. Sci., 59, pp 1-27 (1970).

The success of the coupling reaction at each stage of the synthesis, if performed manually, is preferably monitored by the ninhydrin reaction, as described by E. Kaiser et al., Anal. Biochem. 34, 595 (1970). The coupling reactions can be performed automatically, as on a Beckman 990 automatic synthesizer, using a program such as that reported in Rivier et al. Biopolymers, 1978, 17, pp 1927-1938.

After completing the growing peptide chains, the protected peptide resin is treated with liquid hydrofluoric acid to deblock and release the peptides from the support. For preparing an amidated peptide, the resin support used in the synthesis is selected to supply a C-terminal amide, after peptide cleavage from the resin. After removal of the hydrogen fluoride, the peptide is extracted into 1M acetic acid solution and lyophilized.

The peptide can be isolated by an initial separation by gel filtration, to remove peptide dimers and higher molecular weight polymers, and also to remove undesired salts.

Test and tolerogenic peptide sequences need not correspond exactly to the amino acid sequence of the agent infecting the host from which the PBMCs to be tolerised are derived. It is well known that proteins from wild type isolates of infectious agents often contain differences relative to the sequences of reference isolates of that agent. However, use of peptides synthesised according to reference sequences will typically provide the desired tolerogenic effects.

In some circumstances, it may be desirable and feasible to use a test or tolerogenic sequence not from the agent infecting the host, but from a related agent, as long as the agents are sufficiently closely related for immunological cross-reactivity to occur, such that the desired tolerance is induced.

Alternatively, it may be desirable deliberately to introduce sequence mutations relative to either a wild type isolate or reference isolate. For example, without wishing to be bound by any particular theory, it is believed that the test/tolerogenic sequences may exert their effects by being presented to T cells with a Tr1 phenotype (3) by antigen presenting cells. Therefore it may be desirable to introduce mutations into a tolerogenic peptide from a given infectious agent in order to enable it to bind to a broader range of MHC molecules, and thus be used to tolerise a larger proportion of a population towards target antigens.

Therefore test or tolerogenic peptides may be used which differ from known or wild type sequences for the corresponding region of the infectious agent protein, as long as they retain sufficient tolerogenic capability. This can readily be determined by use of the methods of the present invention.

Variant peptides can be produced by a mixture of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining peptide conformation. Also included are variants having non-conservative substitutions. As is well known to those skilled in the art, substitutions to regions of a peptide which are not critical in determining its conformation may not greatly affect its activity because they do not greatly alter the peptide's three dimensional structure, and so may not affect the desired activity, e.g. MHC binding. In regions which are critical in determining the peptides conformation or activity such changes may confer advantageous properties on the polypeptide. Indeed, changes such as those described above may confer slightly advantageous properties on the peptide e.g. altered stability or specificity.

Generally variant peptides may be extended at the N- or C-termini, and the C-terminus may be amidated or have a free acid form.

A peptide which is an amino acid sequence variant will generally share at least about 50%, 60%, 70%, 80%, 90% or more sequence identity with a wild type or reference sequence from the relevant infectious agent. In this connection, “sequence identity” means strict amino acid identity between the sequences being compared. Once an amino acid substitution or other modification is made as described above, the peptide is screened for the requisite tolerogenic activity, as described above.

As described above, compositions of the present invention may comprise, in addition to the tolerogenic peptide sequences and optionally target antigens, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included as required.

As the compositions of the present invention comprise peptides as active agents, they will typically be delivered by other routes, e.g. by intravenous, cutaneous or subcutaneous injection, or injection at the site of affliction, when the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. For delayed release, the active agents, e.g. tolerogenic peptide sequences and target antigens, may be included in a pharmaceutical composition for formulated for slow release, such as in microcapsules formed from biocompatible polymers or in liposomal carrier systems according to methods known in the art.

For continuous release of peptides, the peptides may be covalently conjugated to a water soluble polymer, such as a polylactide or biodegradable hydrogel derived from an amphipathic block copolymer, as described in U.S. Pat. No. 5,320,840. Collagen-based matrix implants, such as described in U.S. Pat. No. 5,024,841, are also useful for sustained delivery of peptide therapeutics. Also useful, particularly for subdermal slow-release delivery, is a composition that includes a biodegradable polymer that is self-curing and that forms an implant in situ, after delivery in liquid form. Such a composition is described, for example in U.S. Pat. No. 5,278,202.

Thus in a further aspect, the present invention provides a pharmaceutical composition comprising a tolerogenic peptide-encoding nucleic acid molecule and its use in methods of therapy or diagnosis. The composition may further comprise a target antigen-encoding nucleic acid molecule, which may be contiguous with the tolerogenic peptide-encoding nucleic acid molecule.

In a further aspect, the present invention provides a pharmaceutical composition comprising one or more tolerogenic peptide sequences as defined above and its use in methods of therapy or diagnosis. The composition may further comprise one or more target antigens.

In further aspects, the present invention provides the above described tolerogenic peptide sequences and encoding nucleic acid molecules for use in the preparation of medicaments for therapy.

Peptides may preferably be administered by transdermal iontophoresis. One particularly useful means for delivering compounds is transdermal delivery. This form of delivery can be effected according to methods known in the art. Generally, transdermal delivery involves the use of a transdermal “patch” which allows for slow delivery of compound to a selected skin region. Such patches are generally used to provide systemic delivery of compound. Examples of transdermal patch delivery systems are provided by U.S. Pat. No. 4,655,766 (fluid-imbibing osmotically driven system), and U.S. Pat. No. 5,004,610 (rate controlled transdermal delivery system).

For transdermal delivery of peptides, transdermal delivery may preferably be carried out using iontophoretic methods, such as described in U.S. Pat. No. 5,032,109 (electrolytic transdermal delivery system), and in U.S. Pat. No. 5,314,502 (electrically powered iontophoretic delivery device).

For transdermal delivery, it may be desirable to include permeation enhancing substances, such as fat soluble substances (e.g., aliphatic carboxylic acids, aliphatic alcohols), or water soluble substances (e.g., alkane polyols such as ethylene glycol, 1,3-propanediol, glycerol, propylene glycol, and the like). In addition, as described in U.S. Pat. No. 5,362,497, a “super water-absorbent resin” may be added to transdermal formulations to further enhance transdermal delivery. Examples of such resins include, but are not limited to, polyacrylates, saponified vinyl acetate-acrylic acid ester copolymers, cross-linked polyvinyl alcohol-maleic anhydride copolymers, saponified polyacrylonitrile graft polymers, starch acrylic acid graft polymers, and the like. Such formulations may be provided as occluded dressings to the region of interest, or may be provided in one or more of the transdermal patch configurations described above.

In other treatment methods, the modulators may be given orally or by nasal insufflation, according to methods known in the art. For administration of peptides, it may be desirable to incorporate such peptides into microcapsules suitable for oral or nasal delivery, according to methods known in the art.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector. The vector could be targeted to the specific cells to be treated, or it could contain regulatory elements which are switched on more or less selectively by the target cells.

A composition may be administered alone or in combination with other treatments, either simultaneously or sequentially dependent upon the condition to be treated.

Specific embodiments of the invention will now be described in more detail, by way of example and not limitation, by reference to the accompanying drawings.

### BRIEF DESCRIPTION OF THE DRAWINGS

FIG. 1 shows cytokine and proliferative responses of PBMC from healthy EBV seropositive and seronegative donors to purified LMP1. Representative results are shown from two EBV seropositive donors (n=10) and two seronegative donors.

FIG. 2 shows cytokine and proliferative responses of PBMC from EBV seropositive donors to a panel of LMP1 peptides. Representative results obtained from one donor (n=20) are shown for cytokine ELISAs (IL-10, IL-4, gamma-IFN) and a proliferation assay. The broken line on each chart shows the minimum level considered to be a positive response.

FIG. 3 shows a summary of the percentage of EBV seropositive donors (n=20) whose PBMC responded to each LMP1 peptide with cytokine secretion (IL-10, IL-4, gamma-IFN) or proliferation. The results were demonstrated to be reproducible by retesting all of the 18 available donors.

FIG. 4 shows flow cytometric analysis (23) of the phenotype of IL-10 synthesizing cells. After gating on CD3+ cells, cultured cells from two EBV seropositive donors (A+B and C+D) were analyzed for expression of CD4 and IL-10, with the % of double positive cells shown in the upper right quadrant of each panel. A+C were obtained from unstimulated cultures and B+D from cells stimulated with peptides P14 (aa 66-85) and P8 (aa 36-55) respectively (shown to induce IL-10 in these donors).

FIG. 5 shows proliferative and γ-IFN responses by PBMC from EBV seropositive, but not seronegative, donors against a mitogen (Con A), a recall antigen (PPD) and a primary antigen (KLH) in the presence and absence of LMP1. Representative results are shown from two EBV seropositive donors (n=10) and two seronegative donors.

FIG. 6 shows that IL-10 inducing LMP1 peptides inhibit proliferative responses by PBMC from EBV seropositive donors against recall antigen (PPD). The white bars show the proliferative and gamma-IFN responses obtained when PBMC from three EBV seropositive donors were stimulated with PPD, either alone, or together with IL-10 inducing LMP1 peptides (P4, 7, 23, 35 for Donor 1, P4 and 22 for Donor 2, and P4, 18 and 31 for Donor 3), or control gamma-IFN inducing LMP1 peptides (P28 for Donor 1, P56 for Donor 2 and P33 for Donor 3). The black bars show the effects of adding a neutralizing anti-IL-10 antibody to duplicate cultures at 0.5 μg/ml.

FIGS. 7 to 10 illustrate the specificity and persistence of LMP-1-induced tolerance. In each Figure, panel (a) shows proliferative, γ-IFN and IL-10 responses obtained when PBMC from a given donor were first stimulated in culture with the mitogen Con A, the recall antigen PPD, or the primary antigen KLH, alone or in combination with purified LMP1. In FIGS. 7a and 9a, stimuli were also administered in combination with an LMP-1-derived peptide. Cells were rested for seven days, washed to remove the antigens, and added to fresh irradiated autologous PBMC as a source of antigen presenting cells. Panel (b) shows the results of restimulating the control cells with each of the three stimuli. Panel (c) shows the results of restimulating the cells originally stimulated in the presence of LMP-1. Panel (d), shown only for FIGS. 7 and 9, shows the results of restimulating cells originally stimulated in the presence of LMP-1-derived peptide. Results are shown for three EBV-seropositive donors (FIGS. 7, 8 and 9) and one seronegative donor (FIG. 10).

FIG. 11 shows that antigen processing is required for the induction of IL-10 secretion by purified LMP1. IL-10 responses are shown when PBMC from an EBV-seropositive donor were stimulated with purified LMP1 or IL-10-inducing LMP1 peptides in the presence or absence of the processing inhibitor chloroquine. Shaded bars show control cultures lacking chloroquine; open bars show those with chloroquine.

FIG. 12 shows that responses to recall antigen (PPD) and allergen (house dust mite allergen—HDM) can be inhibited by both single LMP1 peptides and combinations of peptides. Mix 1 contains LMP1 peptides P4, P14, P18 and P23; Mix 2 contains LMP1 peptides P4, P7, P14 and P32; Mix 3 contains LMP1 peptides P7, P14, P18 and P23. Mixtures of peptides were administered to give a final concentration of 15 μg/ml of each peptide in the assay.

FIG. 13 shows the effects of LMP1 peptides on the responses of PBMC from two donors (panels (a) and (b)) to a selection of antigens and also in a mixed lymphocyte reaction (MLR). Antigens included the autoantigen Rhesus D protein (RhD), alpha3(IV)NC1 collagen, house dust mite allergen (HDM) and Timothy grass pollen (TG), with PPD as a positive control. Peptide mixtures 1 to 3 are as in FIG. 12.

FIG. 14 shows that tolerance induced to the allergens HDM and TG by LMP1 is antigen specific and persists in the absence of LMP1 peptide. Protocols were as described above for FIGS. 7 to 10. Panel (a) shows primary stimulation with antigen/allergen alone and with LMP1 peptides; panel (b) shows the effect of restimulating the HDM and TG-treated cells with HDM, TG or PPD.

FIG. 15 shows that LMP1 peptides can be used to inhibit the response to the autoantigen RhD in PBMC from a patient with autoimmune haemolytic anaemia.

FIG. 16 shows that LMP1 peptides can be used to inhibit the response to allergens HDM and TG in PBMC from a patient with hay fever and asthma. Panel (a) shows primary stimulation with allergen or antigen alone and with LMP1 peptides. Results from restimulation with HDM, TG and PPD are shown in panel (b).

### EXAMPLES

LMP1 induces high levels of IL-10 secretion by PBMC from EBV seropositive but not seronegative donors.

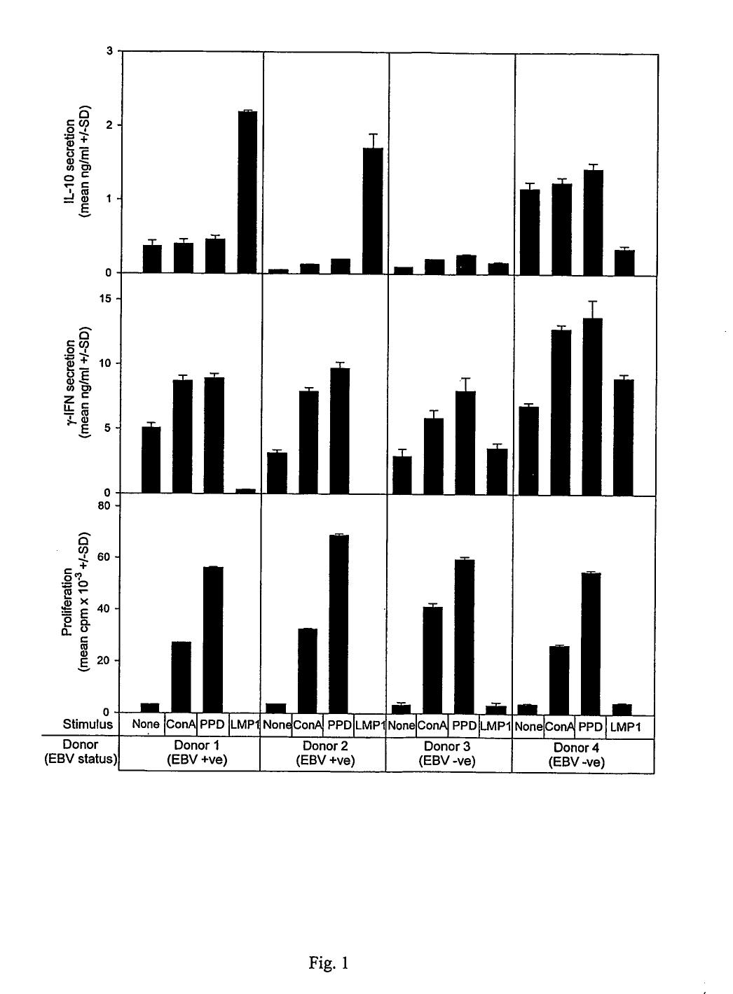

PBMC from ten EBV seropositive donors were tested for the ability to respond to purified LMP1 with either Th cytokine secretion or proliferation. In all seropositive donors, IL-10 was the predominant cytokine measured, with no significant proliferative, gamma-IFN or IL-4 responses. FIG. 1 shows representative results obtained from two seropositive donors. To confirm that the observed responses resulted from previous EBV infection, PBMC from two EBV seronegative donors were tested for responsiveness to the purified LMP1. It can also be seen from FIG. 1 that, in these donors, the LMP1 failed to elicit either IL-10 secretion, or significant proliferative and γ-IFN responses. The results in both donor groups are specific to LMP1, since the T-cell mitogen concanavalin A (Con A) and the control recall antigen Mycobacterium tuberculosis purified protein derivative (PPD) induced responses dominated by proliferation and γ-IFN production, regardless of EBV serological status.

PBMC from EBV Seropositive Donors Respond Strongly to Multiple LMP1 Peptides by Secreting IL-10.

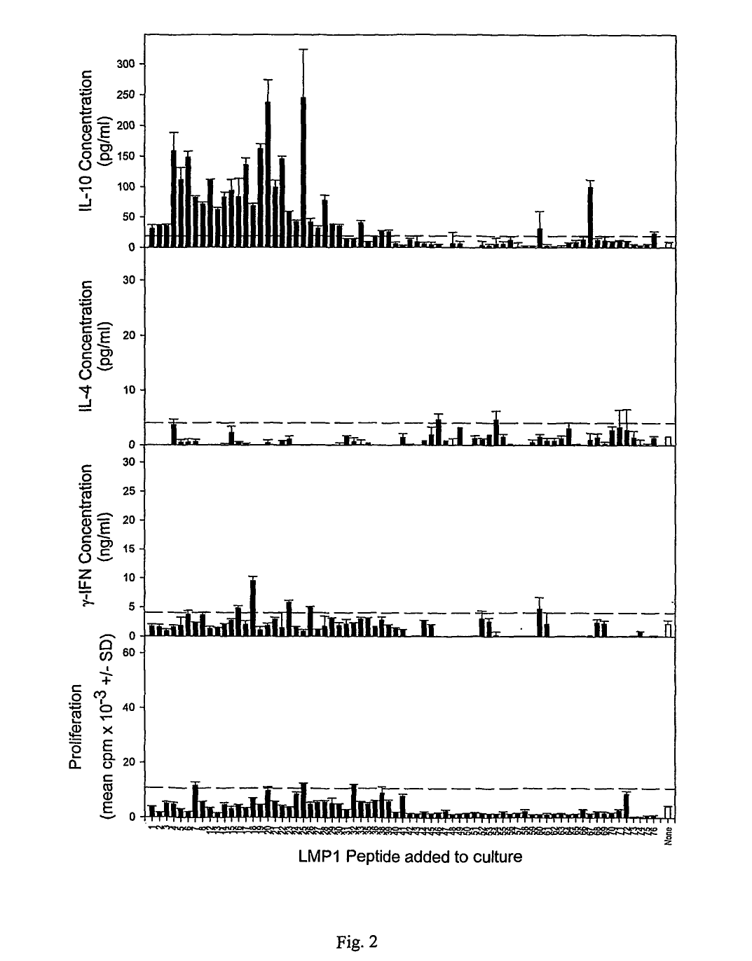

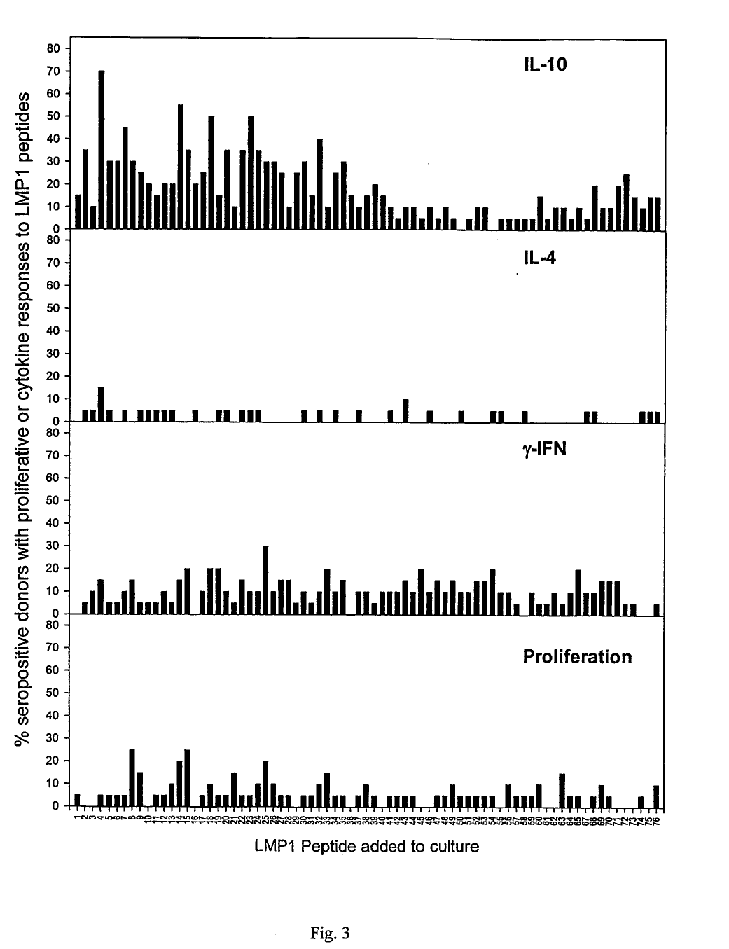

To further characterize the immune response to LMP1, epitopes that induced IL-10 secretion were mapped by screening PBMC from 20 EBV seropositive healthy donors with a panel of synthetic 20-mer peptides spanning the entire sequence of LMP1. Representative results obtained from one donor (FIG. 2) demonstrate that multiple LMP1 peptides induced secretion of high concentrations of IL-10. In contrast, only three peptides induced proliferation, five peptides γ-IFN, two peptides IL-4, and all the latter responses were weak. Similar patterns of responsiveness were found in a total of 20 seropositive donors (summarized in FIG. 3). Strikingly, certain peptides commonly elicited IL-10 responses in different donors (p=2.2.10⁻⁷, Poisson heterogeneity test), with, for example, peptide 4 (aa 16-35) inducing IL-10 in 80% of the individuals. Furthermore, these dominant IL-10 inducing peptides are clustered within the N terminal half of the protein that is rich in binding motifs for many MHC class II molecules (http://imtech.res.in/raghava/propred/index.html; http://www.csd.abdn.ac.uk/˜gjlk/MHC-Thread) (Sturniolo et al. (1999); Singh and Raghava (2001)).

PBMC from four EBV seronegative donors were also screened with the panel of LMP1 peptides. Reactivity was rare in this group, with totals of only nine IL-10, one γ-IFN, one proliferative and no IL-4 responses. Moreover, all these responses were relatively weak (data not shown).

Cells Responding to LMP1 and LMP1 Peptides with IL-10 Secretion are CD3⁺ CD4⁺.

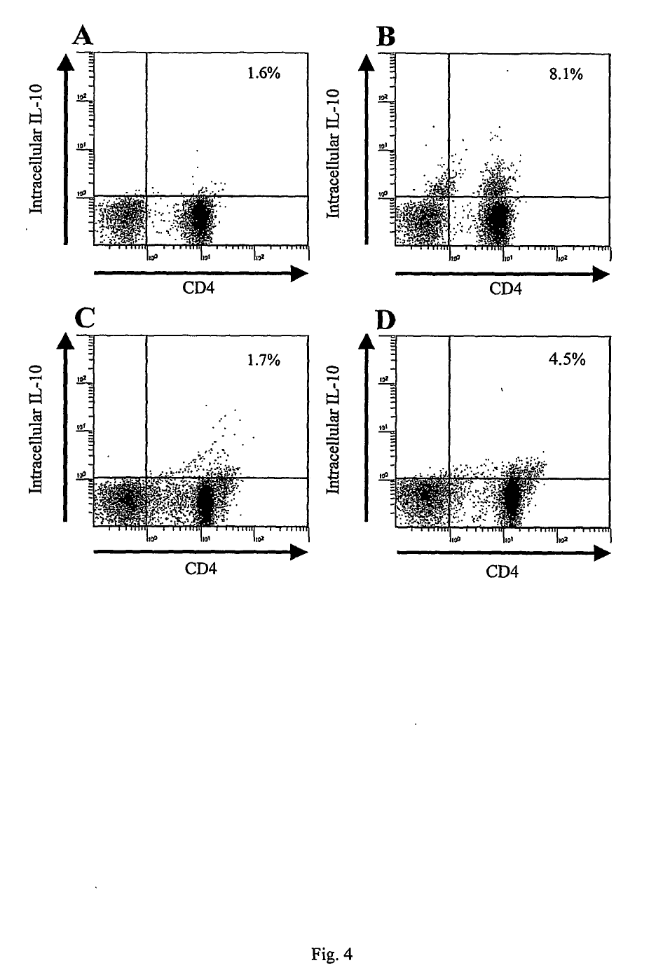

The phenotype of the cells responsible for the IL-10 production was determined by flow cytometry, comparing peptide stimulated and unstimulated cultures from four seropositive individuals. Most IL-10 producing cells bore the CD3 marker for T-cells (mean=83.9%, SD=9.4%) and of these the majority were of the CD4⁺ helper phenotype (mean=90.6%, SD=8.9%) (FIG. 4). Similarly, activated cells, as judged by expression of CD69 and CD71, in the rare cultures proliferating in response to peptides were also CD4+ (data not shown).

LMP1 and LMP1 Peptides Suppress Proliferative and Gamma-IFN Responses by Stimulating IL-10 Secreting Tr1 Cells.

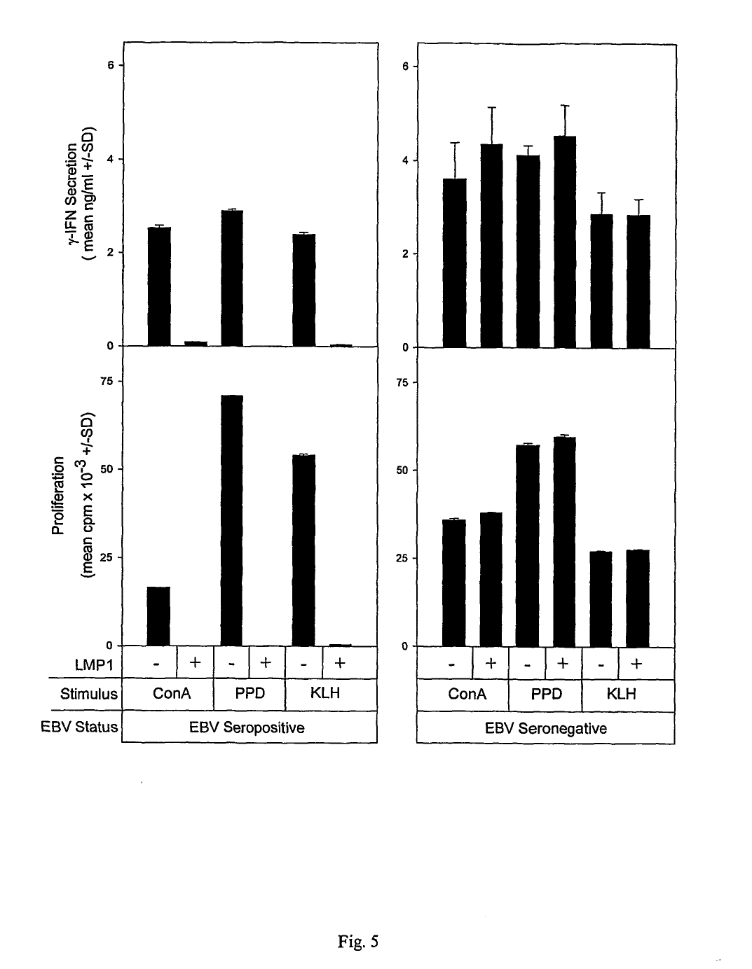

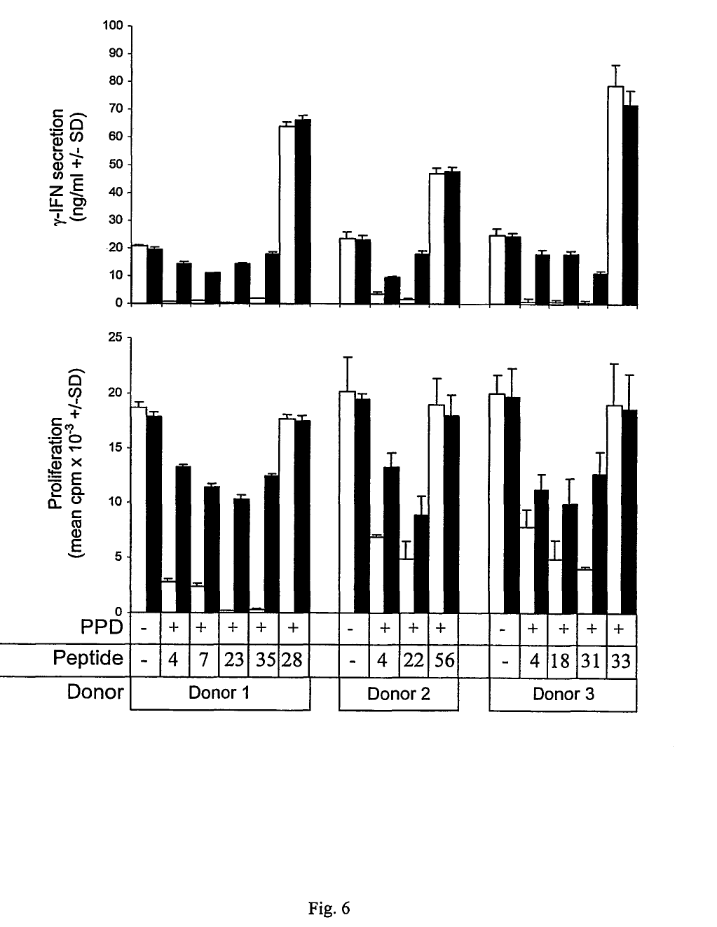

There is evidence that CD4⁺ T-cells biased towards IL-10 secretion, termed Tr1 cells, play an important role in immunoregulation (Groux et al., 1997; Levings and Roncarolo, 2000) and have been shown to inhibit inflammatory responses (Groux et al., 1997; Roncarolo and Levings, 2000). We concluded that the responses to LMP1 and the peptide panel were predominantly mediated by Tr1 cells and sought to confirm that they were capable of mediating suppression. In all ten seropositive donors tested, the addition of LMP1 strongly inhibited proliferative and gamma-IFN responses to the T-cell mitogen Con A, the recall antigen PPD and the primary antigen keyhole limpet hemocyanin (KLH) by 56-99% (FIG. 5). The IL-10 responses to LMP1, and the associated inhibition, were dependent on the donor having been infected with EBV, since no such effects were seen when PBMC were obtained from two control seronegative volunteers (FIG. 5). Results similar to those obtained with purified LMP1 protein were found when IL-10 inducing LMP1 peptides were added to PBMC cultures from five seropositive donors, with suppression of proliferative and gamma-IFN responses to PPD by 41-99% (FIG. 6). In parallel experiments, the peptides also inhibited proliferative and gamma-IFN responses to the mitogen Con A or primary antigen KLH (results not shown). FIG. 6 also demonstrates that the inhibitory effect is dependent on IL-10, since, when LMP1 derived peptides that did not elicit this cytokine were added to PPD-stimulated cultures, no suppression was seen. Furthermore, in cultures treated with anti-IL-10 antibody, the LMP1 peptide mediated suppression was reversed by up to 71% (FIG. 6).

In vitro LNP1 Mediated Suppression is Antigen Specific and Persistent.

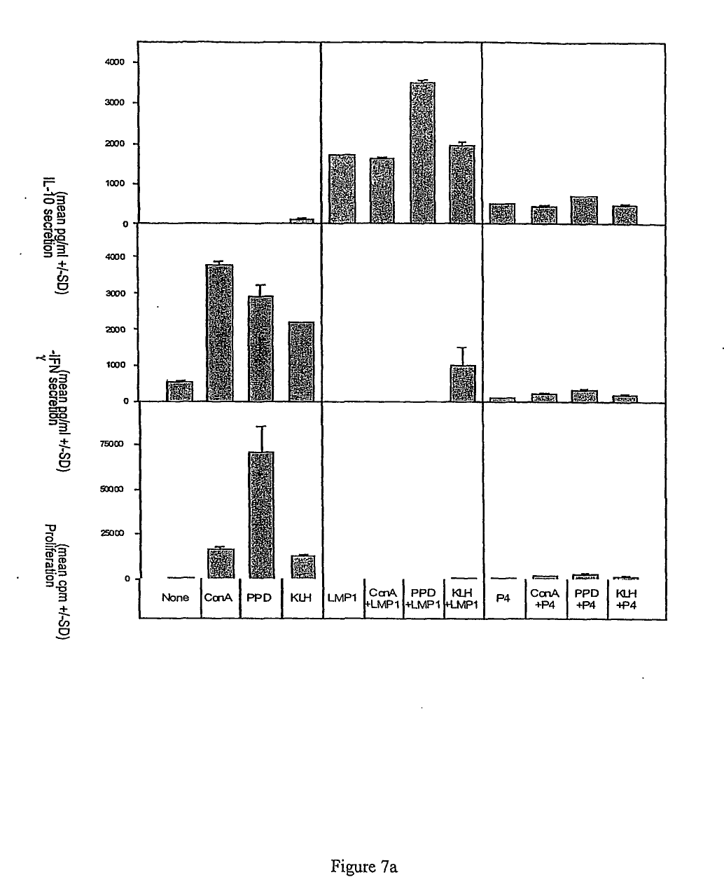

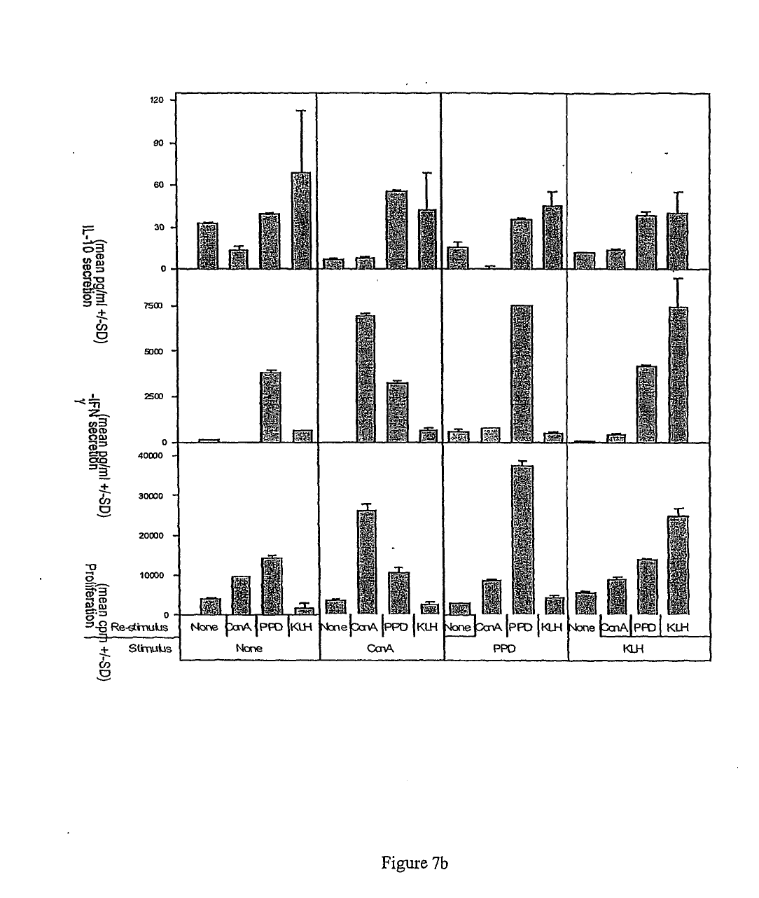

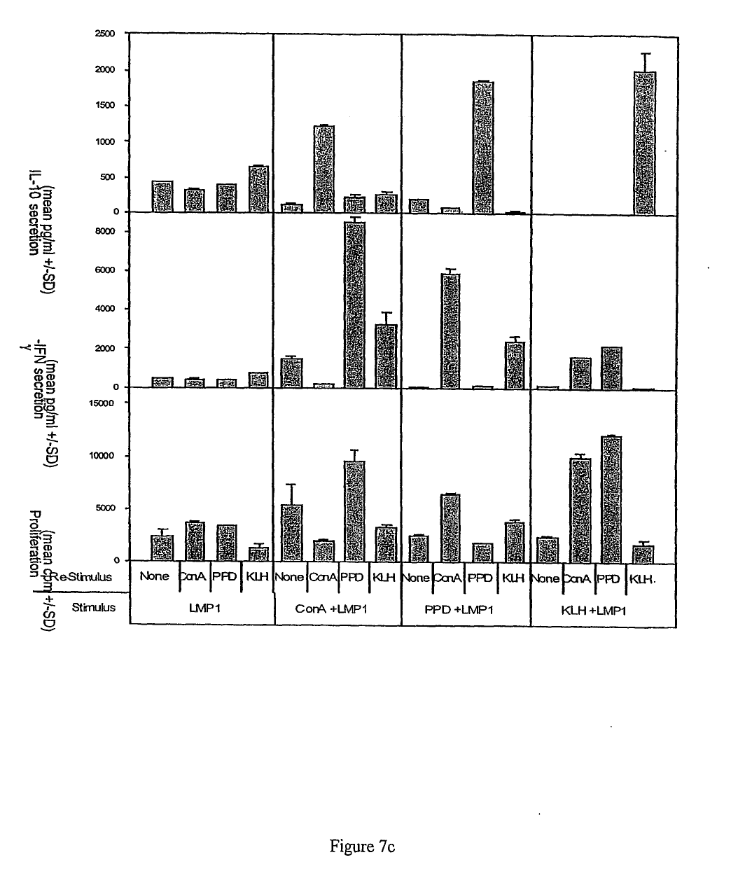

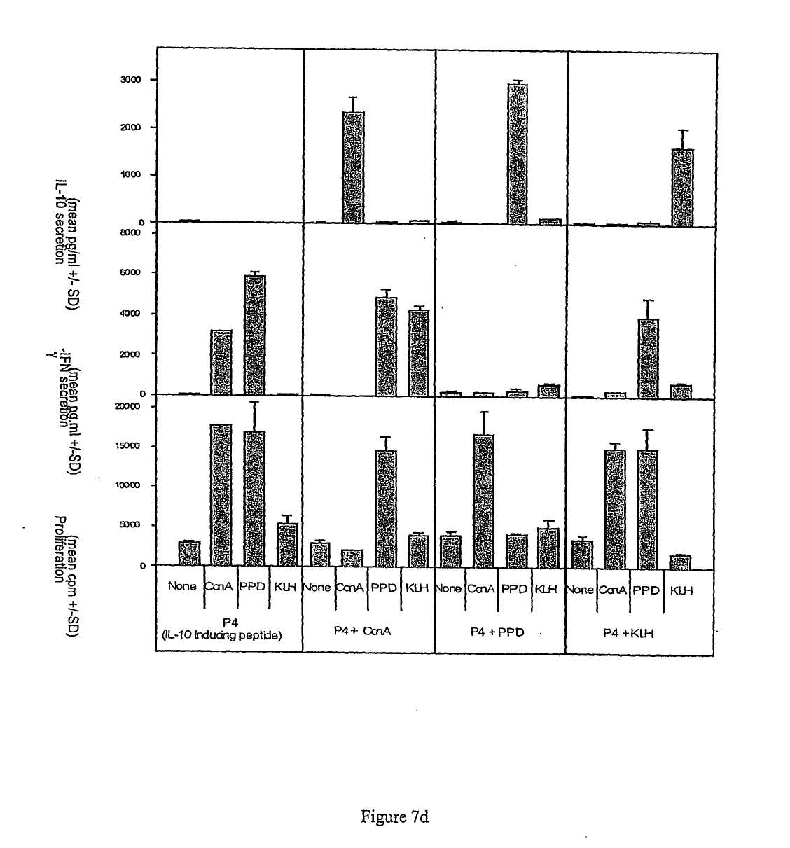

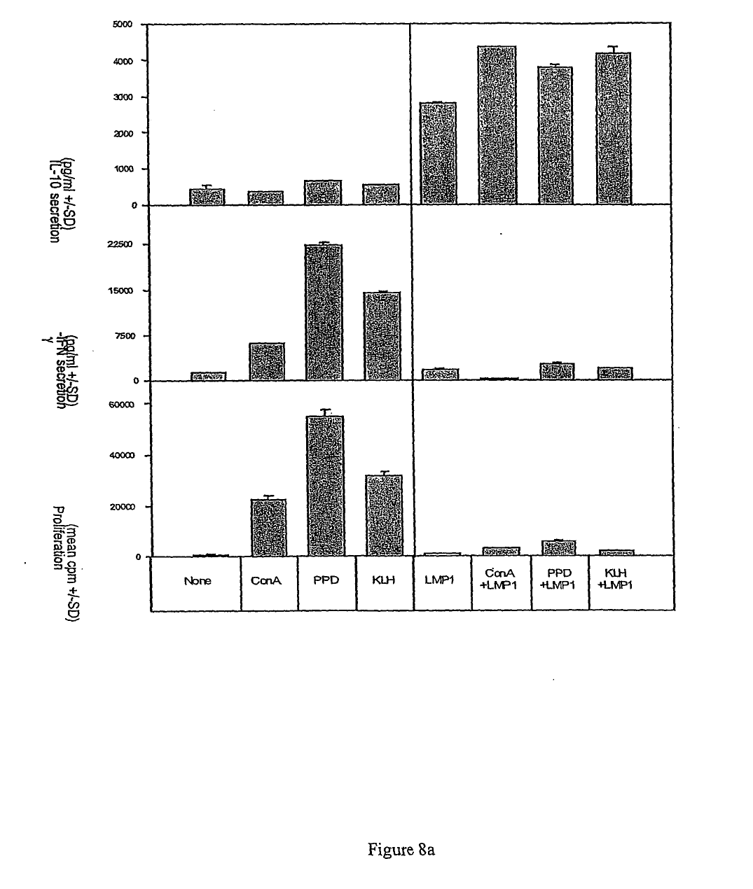

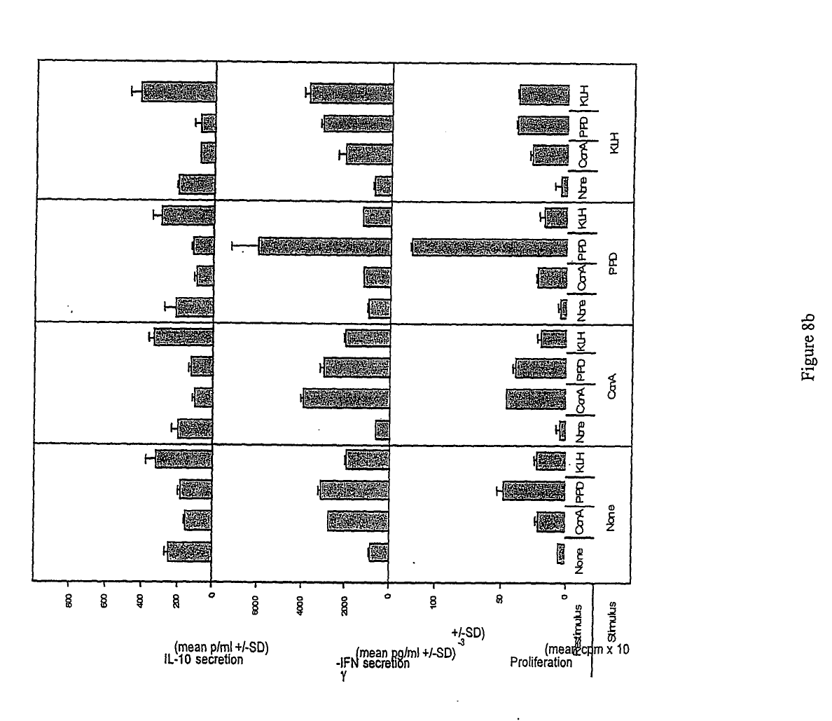

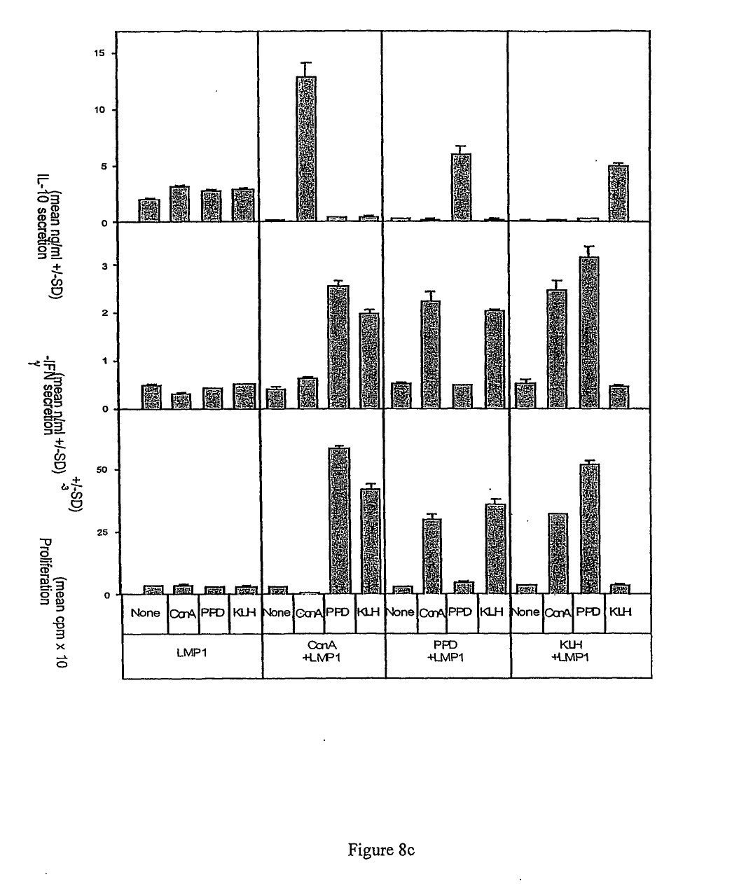

PBMCs from three seropositive donors and one seronegative donor were first stimulated in culture with the mitogen Con A, the recall antigen PPD, or the primary antigen KLH, alone or in combination with purified LMP1, and in two cases in combination with an LMP-1-derived peptide. Subsequently, cells were rested for seven days, washed to remove the antigens, and added to fresh irradiated autologous PBMC as a source of antigen presenting cells (Plebanski et al., 1992). Each group of cells was then restimulated with each stimulus. Results are shown in FIGS. 7 to 10.

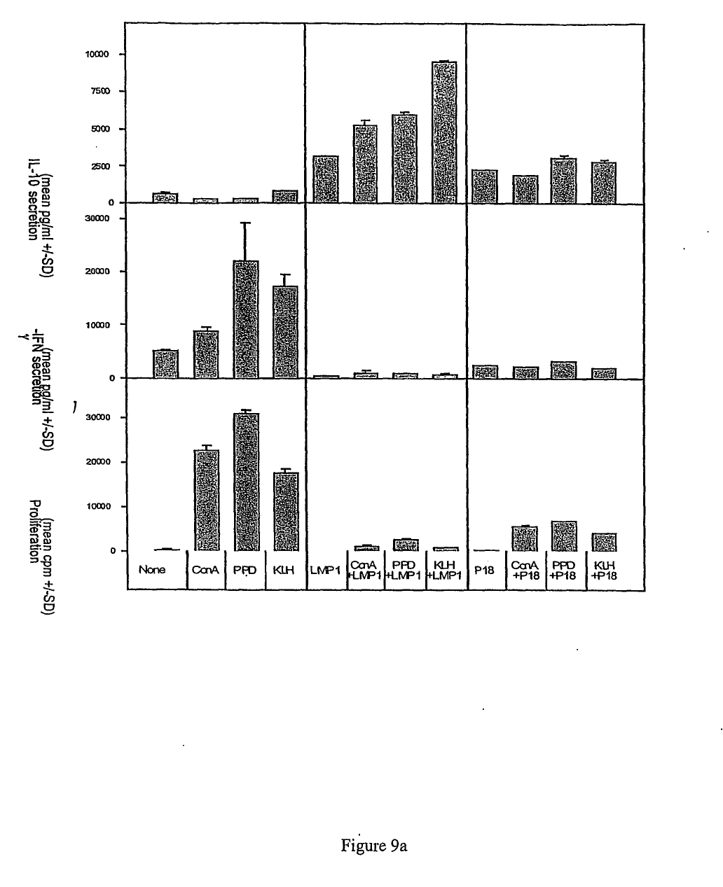

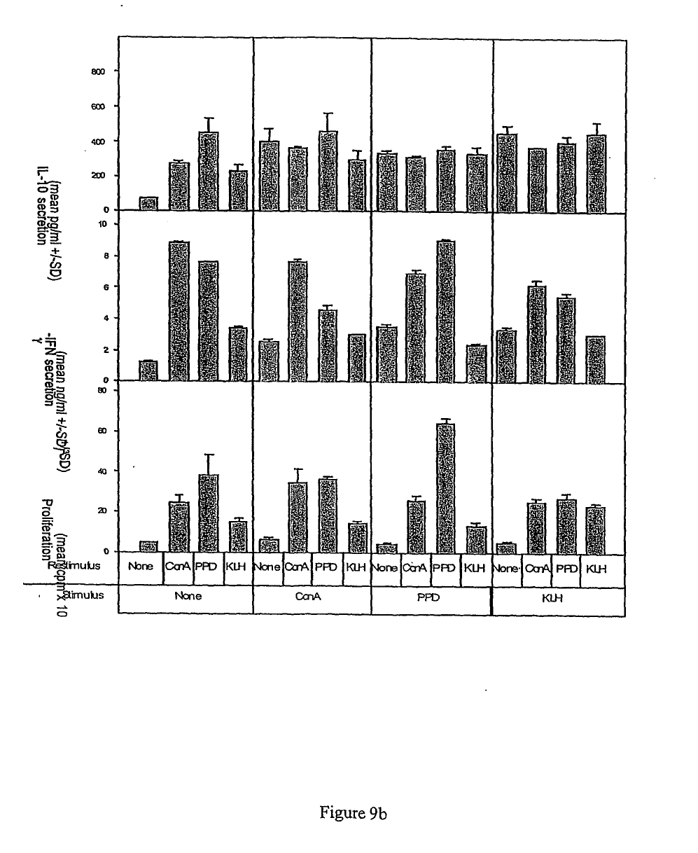

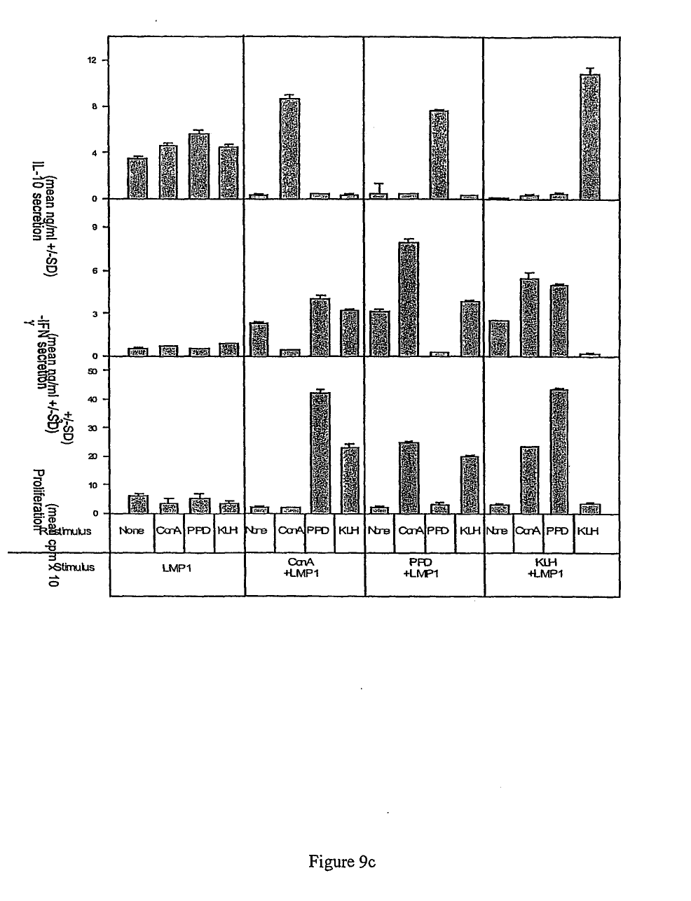

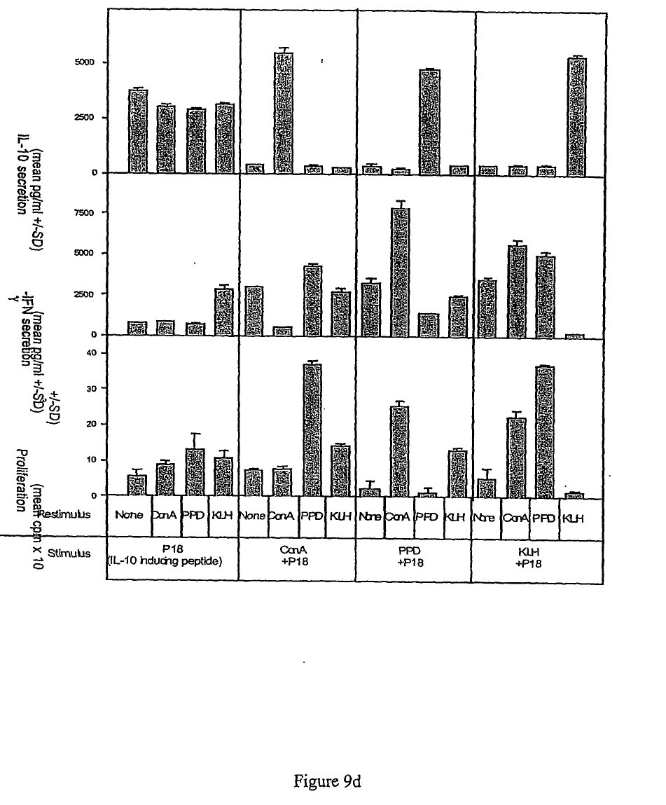

Cells from seropositive donors (FIGS. 7, 8 and 9) which were initially exposed to a stimulus in combination with LMP-1 produced significant IL-10 and low IFN-gamma and proliferative responses. When re-stimulated with the same stimulus, in the absence of LMP-1, these cells still failed to proliferate or express IFN-gamma. However, they retained the capacity to proliferate and produce IL-10 against other stimuli, showing that the cells had been specifically tolerised to the stimulus originally administered in combination with LMP-1. Similar results were obtained for two of the seropositive donors with LMP-1-derived peptides (peptides 4 and 18, shown in FIGS. 7d and 9d respectively). Similar results were also obtained for a further recall antigen, tetanus toxoid (data not shown).

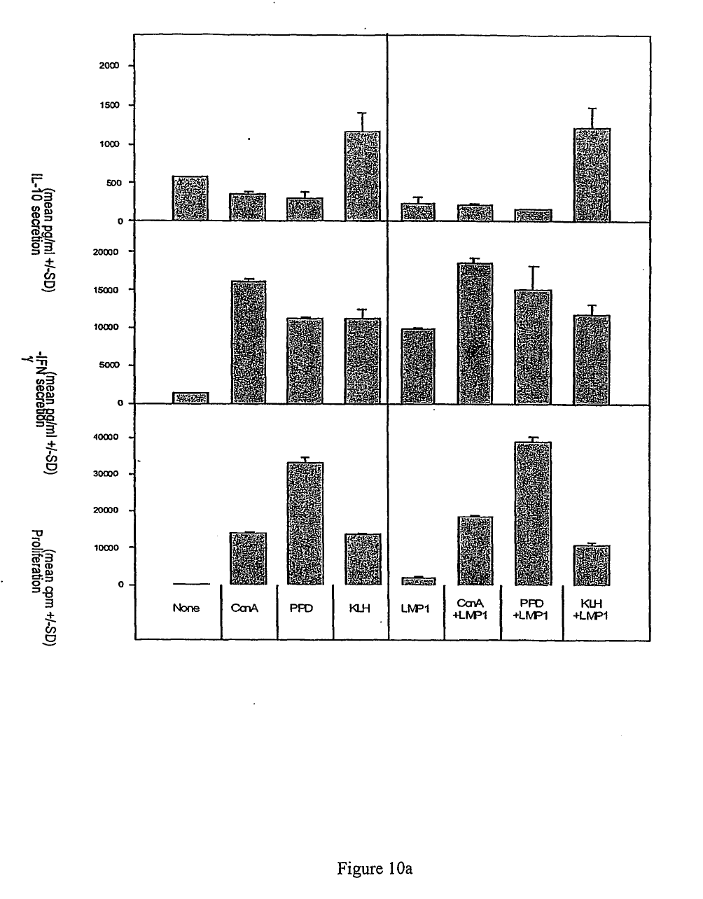

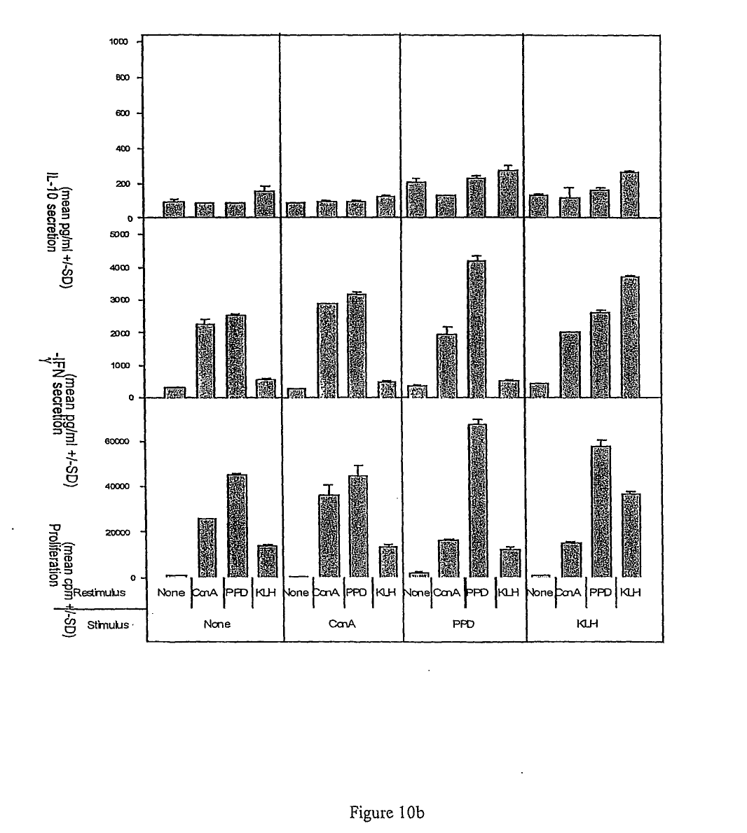

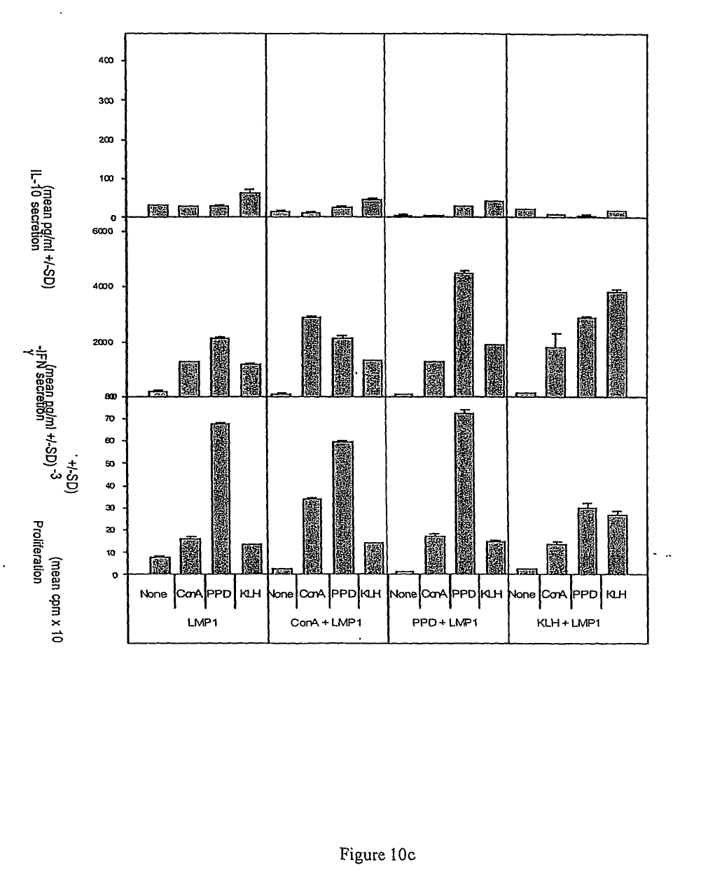

Cells from the seronegative donor (FIG. 10) responded with typical IFN-gamma and proliferative responses to all stimuli regardless of the presence of LMP-1, showing that the induction of tolerance is not an inherent property of the protein, but relies on prior exposure of the cells to EBV.

Thus, it can be hypothesised that the Tr1 response to LMP1 deviates T-cells recognizing a bystander antigen to adopt an anergic, IL-10 secreting phenotype. Such induction of anergy specific for other viral antigens that are co-expressed with LMP1 may be important in the maintenance of EBV latency.

Inhibition of Antigen Processing Prevents IL-10 Secretion Induced by LMP1 Protein, but not Synthetic LMP1 Peptides

The induction of IL-10 secretion from CD4⁺ T cells by the LMP1 peptides suggests that whole LMP1 may induce such responses after the protein has been processed and presented as antigenic peptide fragments by the APC. However, molecules from other pathogens have been shown to induce IL-10, not after processing, but by direct interaction with innate pattern recognition receptors (McGuirk et al., 2002; Mills et al., 2002; Urban et al., 2001).

To investigate the requirement for processing in LMP1-mediated suppression, antigen loading was therefore inhibited by the addition of chloroquine.

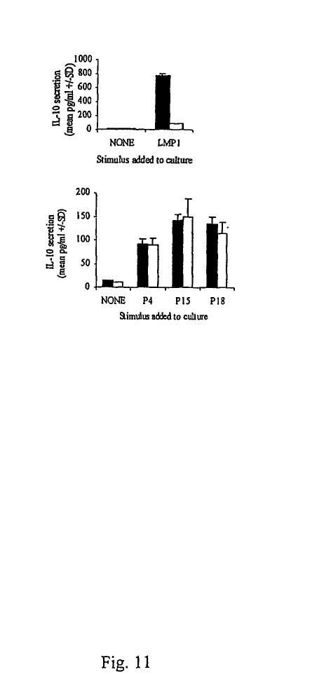

PBMC cultures, with chloroquine-treated or control APC, were stimulated with purified LMP1 or IL-10-inducing LMP1 peptides. The results (FIG. 11) show that inhibition of Ag processing prevents IL-10 secretion induced by purified LMP1, but not by the LMP1 peptides.

Both Th1 and Th2 Responses can be Inhibited by Single LMP1 Peptides and Combinations of Peptides.

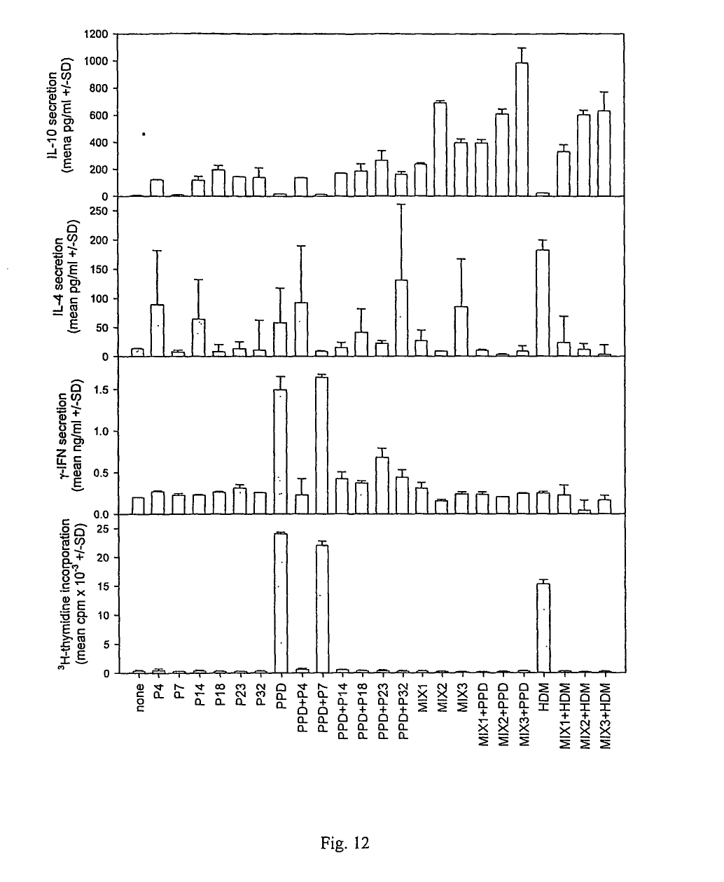

The effects of selected LMP1 peptides and combinations of peptides on responses of seropositive PBMCs to PPD and house dust mite (HDM) allergen were assessed. PPD was chosen as it gives a response representative of a Th1-type response, while HDM was chosen to give a representative pathogenic IL-4-dominated allergic-type Th2 response. Peptide mixtures were chosen to minimise the chances of any given donor failing to produce an IL-10 response when stimulated with the mixture. FIG. 12 shows results from one representative donor. As expected, administration of PPD alone provokes proliferation accompanied by IFN-gamma secretion, while HDM alone provokes proliferation and IL-4 secretion. In both cases, though, these reactions were suppressed by all three mixtures of peptides.

LMP1 Peptides Suppress Responses of Normal Individuals to Auto- and Alloantigens and in Mixed Lymphocyte Reations.

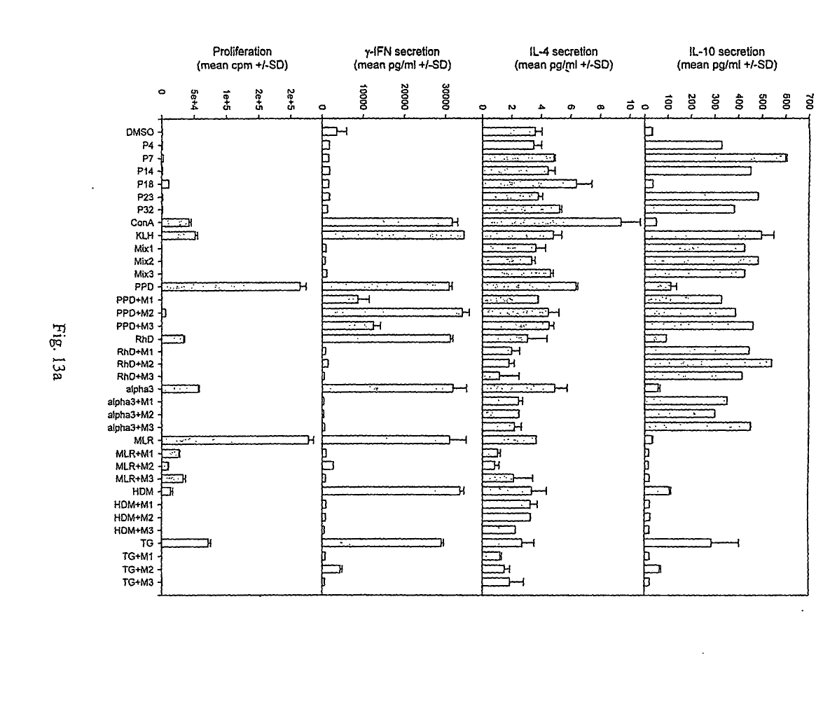

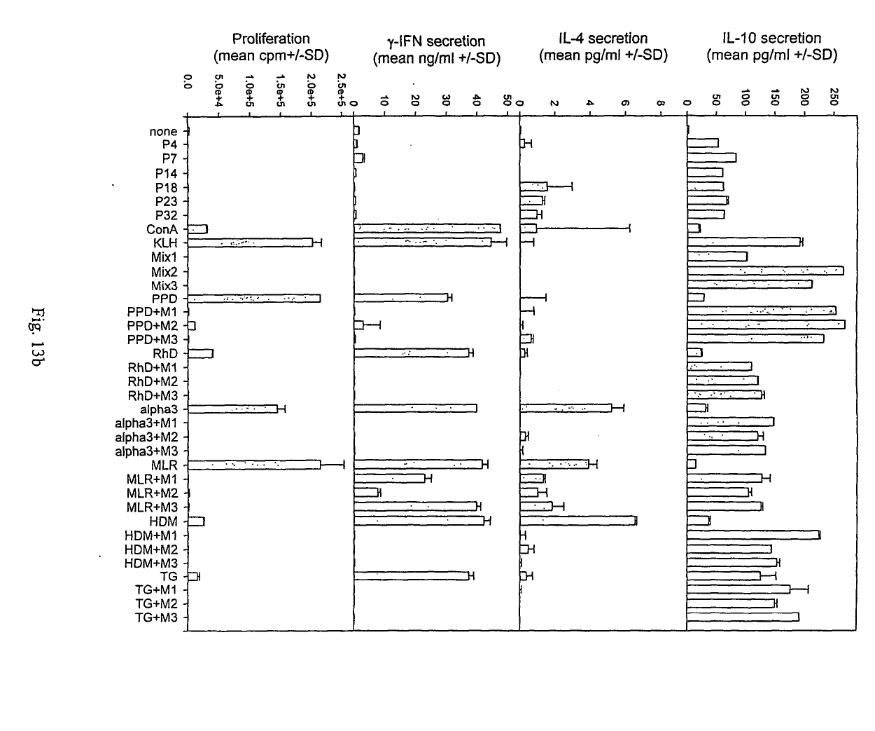

FIG. 13 shows responses of PBMCs from two normal individuals to antigens implicated in auto- and alloimmune responses were investigated. These donors gave the expected reactions to PPD, ConA and KLH (although unusually in this assay stimulation with KLH alone resulted in significant amounts of IL-10 production) which were suppressed by LMP1 peptide mixtures.

The Rhesus D protein (RhD) is the dominant autoantigen in auto- and many allo-immune haemolytic anaemias. Alpha3(IV)NC1 (a3) is a collagen which is the target in Goodpasture's disease. The Th1 responses to both of these antigens of pathogenic significance was also inhibited in these individuals and deviated to IL-10 production.

Use of allogeneic PBMCs as stimulators of a mixed lymphocyte reaction (MLR) with donor cells resulted in massive proliferation and appreciable gamma-interferon secretion. Yet again, the LMP1 peptide mixes were able to profoundly inhibit even these very strong responses. In panel (b), there can be seen to be IL-10 secretion instead. In panel (a), there is slightly less strong inhibition and no significant IL-10 production (it is possible that this is due to an error in sampling the wrong time point). These MLRs serve as a model for mis-matched HLA in transplant situations.

Finally, FIG. 13 shows results obtained with two allergens, Timothy Grass (TG) and House Dust Mite (HDM). In panel (b), from a mildly atopic donor, HDM induces a typical Th2 response with IL-4, appreciable gamma interferon/proliferation and relatively low amounts of IL-10. The peptide mixes abolish the Th2 responses and replace them with IL-10. TG provokes a mixture of ‘tolerant’ and Th1 responses. The peptide mixes abolish the latter and augment the former. In panel (a), there is already tolerance to both antigens, although the mixes are able to inhibit the proliferative and gamma interferon components. There is less IL-10 secreted, the reasons for which are unclear. Thus, once again, it has been demonstrated that Th2 responses can be inhibited by LMP1 peptides.

Suppression of Th2 Responses to Allergens is Antigen-Specific and Persistent

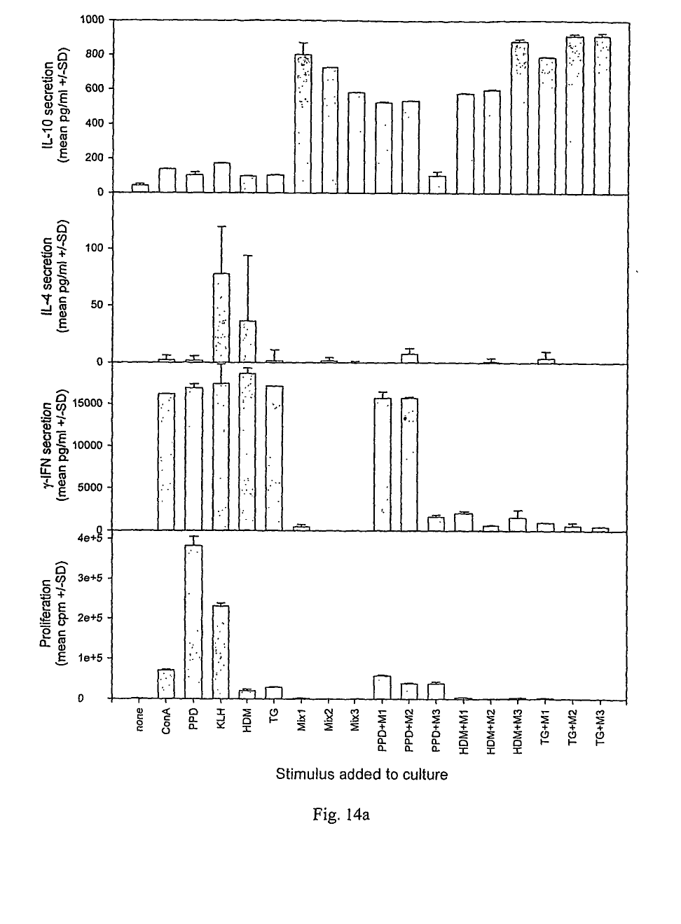

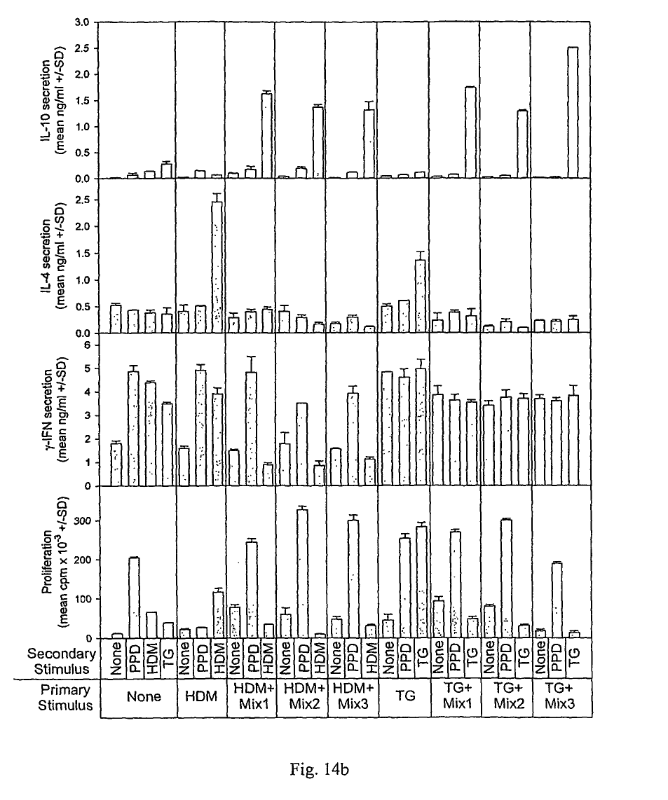

PBMCs from a normal donor were stimulated with the allergens HDM and TG alone and in combination with peptide mixtures 1 to 3. Various other antigens were used as controls. Responses are shown in FIG. 14, panel (a). Panel (b) shows the results of restimulation with HDM, TG and PPD. In all cases, responses to the primary stimulus were suppressed but the cells retained the capacity to respond normally to other antigens. Thus suppression of IL-4-driven Th2 responses to allergens is antigen-specific and persists in the absence of LMP1 peptide.

LMP1 peptides can Inhibit Th1-Type Autoimmune Responses in Cells from Affected Individuals

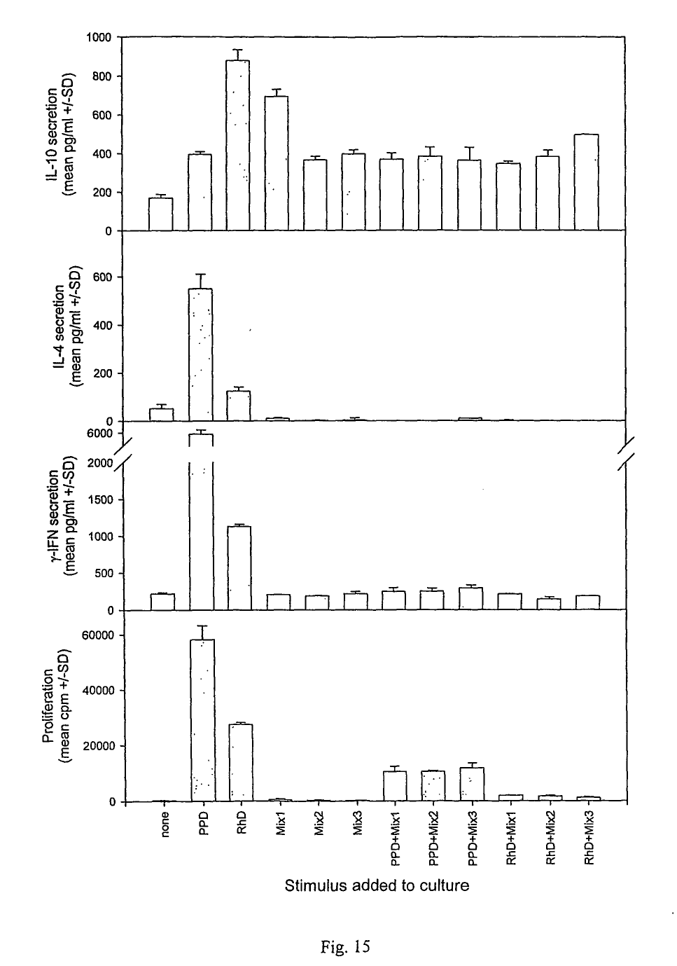

Cells from a patient with autoimmune haemolytic anaemia stimulated with RhD antigen alone gave a strong proliferative response with secretion of IFN-gamma. These Th1 components of the response were profoundly inhibited by all three of the LMP1 peptide mixtures described above (FIG. 15).

LMP1 Peptides can Inhibit Allergic Responses in Cells from Affected Individuals

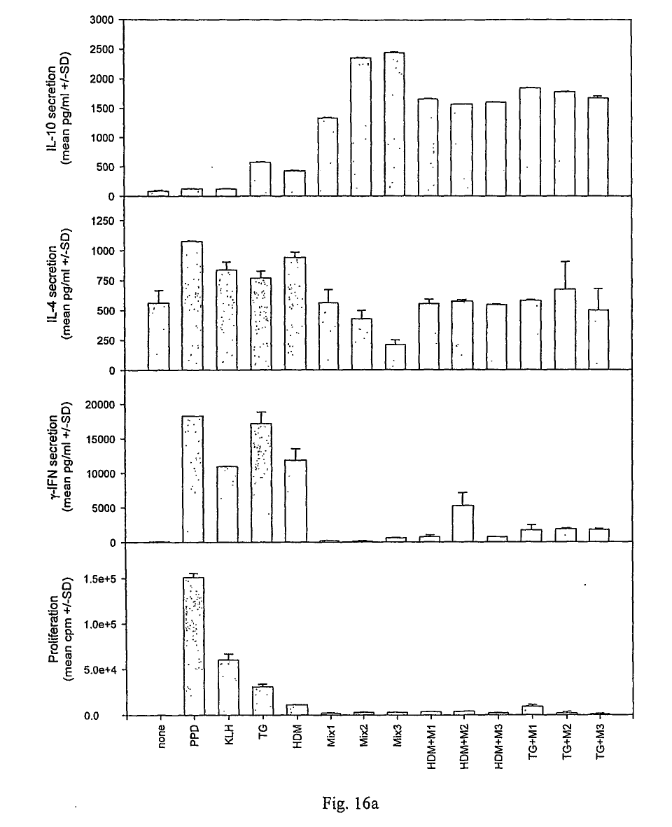

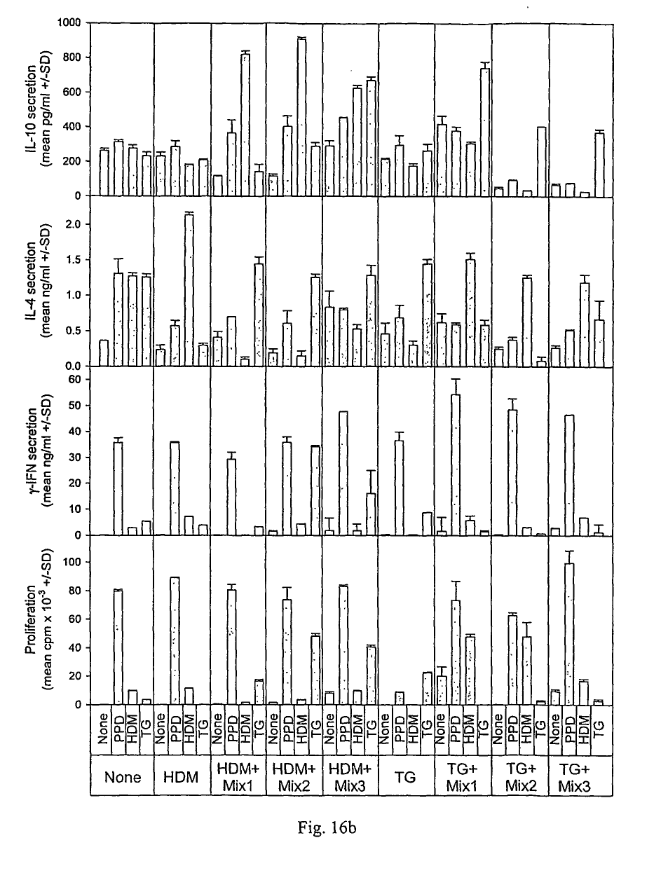

Proliferative and IFN-gamma responses to HDM and TG allergens were reduced almost to background levels by LMP1 peptide mixtures in PBMCs from a patient suffering from hay fever and asthma (FIG. 16a). IL-4 secretion was also inhibited in these cells, although the IL-4 background is very high in the assay shown. Restimulation of these cells with PPD, HDM and TG in the absence of peptide showed again that the cells' responses to the primary stimulus were suppressed but that they retained the capacity to respond to other antigens.

Discussion of Examples

Here a novel mechanism is postulated by which Epstein-Barr virus (EBV), rather than avoiding detection, instead subverts the immune response by stimulating regulatory CD4⁺ T-cells that secrete the inhibitory cytokine interleukin-10 (IL-10). Such regulatory T-cells are well recognized (1-3) but not known to have a role in viral persistence (4-6). Human peripheral blood mononuclear cells (PBMC) from all EBV seropositive, but not seronegative, donors responded to both purified latent membrane protein 1 (LMP1) and the corresponding immunodominant peptides with high levels of IL-10 secretion by CD4⁺ T-cells. These IL-10 responses, characteristic of T regulatory 1 (Tr1) cells, coincided with inhibition of T-cell proliferation and γ-interferon (γ-IFN) secretion induced by both mitogen and recall antigen. The ability of this viral antigen to deviate the immune response towards tolerance is likely to be important in maintaining latency and EBV associated tumors.

This study was prompted by the lack of a protective immune response against LMP1 in individuals with latent EBV infection. The main conclusion from our data is that LMP1 is recognized by the immune system, but that this response is dominated by the induction of high levels of IL-10 secretion by cells with a Tr1 phenotype (Levings and Roncarolo, 2000). Furthermore, this IL-10 response was able to suppress both proliferative and γ-IFN responses against other antigens and polyclonal stimuli, and therefore would be expected to prevent the development of protective Th1 and cytotoxic immunity against LMP1 (Fiorentino et al., 1991). Indeed, such IL-10 secretion is also likely to anergise Th1 responses to other EBV proteins co-expressed both in latent infection and associated tumors. Our demonstration of Tr1 activation provides a mechanism for the previously reported observation that recombinant LMP1 inhibits immune functions including mitogen, antigen and CD3/CD28 stimulated T-cell activation; natural killer cell cytotoxicity; and antigen-induced gamma-IFN secretion (Dukers et al., 2000). One peptide from LMP1, included within the sequence of peptide 7 (aa 31-50) from our panel, was reported (Dukers et al., 2000) to replicate these inhibitory properties, and is identified here as one of many effective Tr1 IL-10 inducers.

A possible explanation for the propensity of LMP1 to elicit IL-10 production by Tr1 cells is that the establishment and maintenance of a suppressive response to LMP1 results from IL-10 ‘conditioning’. Activation of CD4⁺ T-cells in the presence of IL-10 leads to the generation of Tr1 cells (Groux et al., 1997). EBV is one of the viruses that encodes a homologue of this cytokine, viral IL-10 (vIL-10)(Hsu et al, 1990), which is expressed during lytic cycle infection (Hayes et al., 1999). Thus, we propose that, during the development of the immune response to EBV, the presence of vIL-10 deviates the differentiation of LMP1 specific Th cells to favor IL-10 secreting Tr1 cells, a bias which then becomes self-perpetuating. The finding that the regulatory response to LMP1 is limited to EBV seropositive, but not seronegative, donors strongly supports this second explanation.

Numerous methods used by pathogens to avoid clearance by the immune system have been described, dependent on escape, resistance or counterattack (Xu et al., 2001). The induction of a Tr1 response to EBV LMP1 represents a further mechanism of immune evasion. A similar regulatory mechanism that subverts, rather than avoids, immune detection may well be exploited by other pathogens with the ability to maintain chronic infections. This is especially likely for those viruses, such as cytomegalovirus, that also encode a homologue of IL-10 with potent immunosuppressive effects (Spencer et al., 2002), which may also induce a regulatory Tr1 type immune response. The design of strategies to overcome such Tr1 responses should provide an innovative approach to the development of vaccines to prevent or treat EBV associated tumors. Conversely, it may be possible to exploit therapeutically such specific induction of bystander anergy to inhibit pathogenic responses in immune-mediated diseases.

Experimental Procedures

Donors

Blood samples were obtained by venepuncture from a group of healthy volunteers. The donors were classified as EBV seropositive or seronegative by an ELISA for serum anti-EBNA1 IgG, with negative results confirmed by immunofluorescence staining for IgG and IgM anti-viral capsid antibody.

Blood samples were also obtained by venepuncture from a male patient with allergic rhinitis, a female patient with atopic asthma, and a male patient with warm-type idiopathic autoimmune haemolytic anaemia.

Antigens and Mitogen

LMP1 was immunopurified from lysed EBV transformed B cells using the anti-LMP1 antibody CS1-4 (Novocastra Laboratories) conjugated to anti-mouse IgG₁ coated magnetic beads (Biomag, PerSeptive Biosystems).

A panel of 76 20-mer peptides, with 15 amino acid overlaps, was synthesised (Department of Biochemistry, University of Birmingham, UK or University of Bristol, UK), spanning the entire length of the 63 kD EBV LMP1, as determined from the prototype B-cell-derived gene (B95.8) sequence (Hayes et al., 1999). All peptides were used to stimulate cultures at 15 μg/ml, although, as in previous mapping studies (Stott et al., 2000), responses were similar over a wide range of concentrations (4-50 μg/ml). Where mixtures of peptides were used, each peptide was present in the assay at 15 μg/ml.

Peptide sequences are as follows:

| | |
|---|---|
| P1 | MEHDLERGPPGPRRPPRGPP |
| P2 | ERGPPGPRRPPRGPPLSSSL |
| P3 | GPRRPPRGPPLSSSLGLALL |
| P4 | PRGPPLSSSLGLALLLLLLA |
| P5 | LSSSLGLALLLLLLALLFWL |
| P6 | GLALLLLLLALLFWLYIVMS |
| P7 | LLLLALLFWLYIVMSDWTGG |
| P8 | LLFWLYIVMSDWTGGALLVL |
| P9 | YIVMSDWTGGALLVLYSFAL |
| P10 | DWTGGALLVLYSFALMLIII |
| P11 | ALLVLYSFALMLIIIILIIF |
| P12 | YSFALMLIIIILIIFIFRRD |
| P13 | MLIIIILIIFIFRRDLLCPL |
| P14 | ILIIFIFRRDLLCPLGALCI |
| P15 | IFRRDLLCPLGALCILLLMI |
| P16 | LLCPLGALCILLLMITLLLI |
| P17 | GALCILLLMITLLLIALWNL |
| P18 | LLLMITLLLIALWNLHGQAL |
| P19 | TLLLIALWNLHGQALFLGIV |
| P20 | ALWNLHGQALFLGIVLFIFG |
| P21 | HGQALFLGIVLFIFGCLLVL |
| P22 | FLGIVLFIFGCLLVLGIWIY |
| P23 | LFIFGCLLVLGIWIYLLEML |
| P24 | CLLVLGIWIYLLEMLWRLGA |
| P25 | GIWIYLLEMLWRLGATIWQL |
| P26 | LLEMLWRLGATIWQLLAFFL |
| P27 | WRLGATIWQLLAFFLAFFLD |
| P28 | TIWQLLAFFLAFFLDLILLY |
| P29 | LAFFLAFFLDLILLIIALYL |
| P30 | AFFLDLILLIIALYLQQNWW |
| P31 | LILLIIALYLQQNWWTLLVD |
| P32 | IALYLQQNWWTLLVDLLWLL |
| P33 | QQNWWTLLVDLLWLLLFLAI |
| P34 | TLLVDLLWLLLFLAILIWMY |
| P35 | LLWLLLFLAILIWMYYHGQR |
| P36 | LFLAILIWMYYHGQRHSDEH |
| P37 | LIWMYYHGQRHSDEHHHDDS |
| P38 | YHGQRHSDEHHHDDSLPHPQ |
| P39 | HSDEHHHDDSLPHPQQATDD |
| P40 | HHDDSLPHPQQATDDSGHES |
| P41 | LPHPQQATDDSGHESDSNSN |
| P42 | QATDDSGHESDSNSNEGRHH |
| P43 | SGHESDSNSNEGRHHLLVSG |
| P44 | DSNSNEGRHHLLVSGAGDGP |
| P45 | EGRHHLLVSGAGDGPPLCSQ |
| P46 | LLVSGAGDGPPLCSQNLGAP |
| P47 | AGDGPPLCSQNLGAPGGGPD |
| P48 | PLCSQNLGAPGGGPDNGPQD |
| P49 | NLGAPGGGPDNGPQDPDNTD |
| P50 | GGGPDNGPQDPDNTDDNGPQ |
| P51 | NGPQDPDNTDDNGPQDPDNT |
| P52 | PDNTDDNGPQDPDNTDDNGP |
| P53 | DNGPQDPDNTDDNGPHDPLP |
| P54 | DPDNTDDNGPHDPLPQDPDN |
| P55 | DDNGPHDPLPQDPDNTDDNG |
| P56 | HDPLPQDPDNTDDNGPQDPD |
| P57 | QDPDNTDDNGPQDPDNTDDN |
| P58 | TDDNGPQDPDNTDDNGPHDP |
| P59 | PQDPDNTDDNGPHDPLPHSP |
| P60 | NTDDNGPHDPLPHSPSDSAG |
| P61 | GPHDPLPHSPSDSAGNDGGP |
| P62 | LPHSPSDSAGNDGGPPQLTE |
| P63 | SSGSGGDDDDPRGPVQLSYYD |
| P64 | SDSAGNDGGPPQLTEEVENK |
| P65 | NDGGPPQLTEEVENKGGDQG |
| P66 | PQLTEEVENKGGDQGPPLMT |
| P67 | EVENKGGDQGPPLMTDGGGG |
| P68 | GGDQGPPLMTDGGGGBSHDS |
| P69 | PPLMTDGGGGHSHDSGHGGG |
| P70 | DGGGGHSHDSGHGGGDPHLP |
| P71 | HSHDSGHGGGDPHLPTLLLG |
| P72 | GHGGGDPHLPTLLLGSSGSG |
| P73 | DPHLPTLLLGSSGSGGDDDD |
| P74 | TLLLGSSGSGGDDDDPHGPV |
| P75 | LFLAILIWMYYHGQRHSDEH |

A similar panel of peptides was prepared from LMP2 having sequences as follows:

| | |
|---|---|
| P1′ | MGSLEMVPMGAGPPSPGGDP |
| P2′ | MVPMGAGPPSPGGDPDGYDG |
| P3′ | AGPPSPGGDPDGYDGGNNSQ |
| P4′ | PGGDPDGYDGGNNSQYPSAS |
| P5′ | DGYDGGMNSQYPSASGSSGN |
| P6′ | GNNSQYPSASGSSGNTPTPP |
| P7′ | YPSASGSSGNTPTPPMDEER |
| P8′ | GSSGNTPTPPNDEERESNEE |
| P9′ | TPTPPNDEERESNEEPPPPY |
| P10′ | NDEERESNEEPPPPYEDPYW |
| P11′ | RESNEEPPPPYEDYWGNGD |
| P12′ | EPPPPYEDPYWGNGDRHSDY |
| P13′ | YEDPYWGNGDRHSDYQPLGT |
| P14′ | WGNGDRHSDYQPLGTQDQSL |
| P15′ | RHSDYQPLGTQDQSLYLGLQ |
| P16′ | QPLGTQDQSLYLGLQHDGND |
| P17′ | QDQSLYLGLQHDGNDGLPPP |
| P18′ | LGLQHDGNDGLPPPPYSPRD |
| P19′ | DGNDGLPPPPYSPRDDSSQH |
| P20′ | LPPPPYSPRDDSSQHIYEEA |
| P21′ | PPYSPRDDSSQHIYEEAGRG |
| P22′ | RDDSSQHIYEEAGRGSMNPV |
| P23′ | QHIYEEAGRGSMNPVCLPVI |
| P24′ | EAGRGSMMPVCLPVIVAPYL |
| P25′ | SMNPVCLPVIVAPYLFWLAA |
| P26′ | CLPVIVAPYLFWLAAIAASC |
| P27′ | VAPYLFWLAAIAASCFTASV |
| P28′ | FWLAAIAASCFTASVSTVVT |
| P29′ | IAASCFTASVSTVVTATGLA |
| P30′ | FTASVSTVVTATGLALSLLL |
| P31′ | STVVTATGLALSLLLLAAVA |
| P32′ | ATGLALSLLLLAAVASSYAA |
| P33′ | LSLLLLAAVASSYAAAQRKL |
| P34′ | LAAVASSYAAAQRKLLTPVT |
| P35′ | SSYAAAQRKLLTPVTVLTAV |
| P36′ | AQRKLLTPVTVLTAVVTFFA |
| P37′ | LTPVTVLTAVVTFFAICLTW |
| P38′ | VLTAVVTFFAICLTWRIEDP |
| P39′ | VTFFAICLTWRIEDPPFNSL |
| P40′ | ICLTWRIEDPPFNSLLFALL |
| P41′ | RIEDPPFUSLLFALLAAAGG |
| P42′ | PFNSLLFALLAAAGGLQGIY |
| P43′ | LFALLAAAGGLQGIYVLVML |
| P44′ | AAAGGLQGIYVLVMLVLLIL |
| P45′ | LQGIYVLVMLVLLILAYRRR |
| P46′ | VLVMLVLLILAYRRRWRRLT |
| P47′ | VLLILAYRRRWRRLTVCGGI |
| P48′ | AYRRRWRRLTVCGGIMFLAC |
| P49′ | WRRLTVCGGIMFLACVLVLI |
| P50′ | VCGGIMFLACVLVLIVDAVL |
| P51′ | MFLACVLVLIVDAVLQLSPL |
| P52′ | VLVLIVDAVLQLSPLLGAVT |
| P53′ | VDAVLQLSPLLGAVTVVSMT |
| P54′ | QLSPLLGAVTVVSMTLLLLA |
| P55′ | LGAVTVVSMTLLLLAFVLWL |
| P56′ | VVSMTLLLLAFVLWLSSPGG |
| P57′ | LLLLAFVLWLSSPGGLGTLG |
| P58′ | FVLWLSSPGGLGTLGAALLT |
| P59′ | SSPGGLGTLGAALLTLAAAL |
| P60′ | LGTLGAALLTLAAALALLAS |
| P61′ | AALLTLAAALALLASLILGT |
| P62′ | LAAALALLASLILGTLNLTT |
| P63′ | ALLASLILGTLNLTTMFLLM |
| P64′ | LILGTLNLTTMFLLMLLWTL |
| P65′ | LNLTTMFLLMLLWTLVVLLI |
| P66′ | MFLLMLLWTLVVLLICSSCS |
| P67′ | LLWTLVVLLICSSCSSCPLS |
| P68′ | VVLLICSSCSSCPLSKILLA |
| P69′ | CSSCSSCPLSKILLARLFLY |
| P70′ | SCPLSKILLARLFLYALALL |
| P71′ | KILLARLFLYALALLLLASA |
| P72′ | RLFLYALALLLLASALIAGG |
| P73′ | ALALLLLASALIAGGSILQT |
| P74′ | LLASALIAGGSILQTNFKSL |
| P75′ | LIAGGSILQTNFKSLSSTEF |
| P76′ | SILQTNFKSLSSTEFIPNLF |
| P77′ | NFKSLSSTEFIPNLFCMLLL |
| P78′ | SSTEFIPNLFCMLLLIVAGI |
| P79′ | IPNLFCMLLLIVAGILFILA |
| P80′ | CMLLLIVAGILFILAILTEW |
| P81′ | IVAGILFILAILTEWGSGNR |
| P82′ | LFILAILTEWGSGNRTYGPV |
| P83′ | ILTEWGSGNRTYGPVFMCLG |
| P84′ | GSGNRTYGPVFMCLGGLLTM |
| P85′ | FMCLGGLLTMVAGAVWLTVM |
| P86′ | GLLTMVAGAVMLTVMSNTLL |
| P87′ | VAGAVWLTVMSNTLLSAWIL |
| P88′ | WLTVMSNTLLSAWILTAGFL |
| P89′ | SNTLLSAWILTAGFLIFLIG |
| P90′ | SAWILTAGFLIFLIGFALFG |
| P91′ | TAGFLIFLIGFALFGVIRCC |
| P92′ | IFLIGFALFGVIRCCRYCCY |
| P93′ | FALFGVIRCCRYCCYYCLTL |
| P94′ | VIRCCRYCCYYCLTLESEER |
| P95′ | RYCCYYCLTLESEERPPTPY |
| P96′ | YYCLTLESEERPPTPYRNTV |

The control antigen mycobacterial PPD (Statens Seruminstitut), the T-cell mitogen Con A (Sigma), and the primary antigen keyhole limpet hemocyanin (KLH) (Calbiochem) were each used to stimulate cultures at 10 μg/ml. PPD readily provokes recall T-cell responses in vitro (Plebanski et al., 1992), since most UK citizens have been immunised with Bacille Calmette-Guérin (BCG).

RhD protein was prepared as described in Hall, A. M., Ward, F. J., Vickers, M. A., Stott, L-M., Urbaniak, S. J. & Barker, R. N. (2002). Interleukin-10 mediated regulatory T-cell responses to epitopes on a human red blood cell autoantigen. Blood 100:4529-4536. RhD protein was added to cultures at an estimated concentration of 5 μg/ml. Timothy grass pollen extract and house dust mite (Dermatophagoides pteronyssinus) extract (both International Standards) were obtained from National Institute for Biologicals and Controls, Potters Bar, UK, and added to cultures at 2,500 IU/ml. Recombinant α3(IV)NC1 was prepared as previously described (Ryan, J. J., Mason, P. J., Pusey, C. D., Turner, N. Recombinant alpha-chains of type IV collagen demonstrate that the amino terminal of the Goodpasture autoantigen is crucial for antibody recognition. Clin Exp Immunol. 113:17-27, 1998) and used to stimulate cultures at 10 μg/ml.

Cell Proliferation and Cytokine Assays

As described elsewhere (Stott et al., 2000), PBMC were separated from fresh blood samples by density gradient centrifugation and cultured in 1 ml volumes at a concentration of 1.25×10⁶ cells/ml. Cellular proliferation was estimated from the incorporation of H-thymidine in triplicate 100 μl aliquots taken from the wells five days after antigen stimulation, when recall T-cell responses are maximal (Plebanski et al., 1992). Proliferation results are presented as the mean counts per minute (CPM)+/−SD of the triplicate samples, or as stimulation index (SI), expressing the ratio of mean CPM in stimulated versus unstimulated control cultures. An SI>3 with CPM>1000 is interpreted as representing a significant positive response (Devereux et al., 2000). Production of the Th cytokines γ-IFN, IL-4 and IL-10 was assessed in duplicate 100 μl aliquots taken five days after stimulation of the cultures, using a sensitive cellular ELISA (Devereux et al., 2000). Cytokine responses over twice the production in unstimulated cultures were considered positive (Devereux et al., 2000).

Characterisation of Responding Cells

The phenotypes of cultured cells that proliferate or secrete cytokine in response to antigen were determined by flow cytometry. Aliquots of PBMC were taken from responding cultures and stained with anti-CD3 phycoerythrin-Texas Red®-x and anti-CD4 fluorescein isothiocyanate, with anti-CD25 phycoerythrin-cyanin 5.1 (all Beckman Coulter) in some experiments. Activated cells in proliferating cultures were identified using anti-CD71-phycoerythrin (PE) or anti-CD69-PE (both Beckman Coulter). Cells synthesising IL-10 were labelled by incubating with anti-IL-10-PE (Pharmingen) after inhibition of protein secretion with Brefeldin A (Sigma) and permeabilisation with Intraprep™ (Beckman Coulter). Stained cells were analysed using an EPICS XL cytometer (Beckman Coulter) and Expo v2 analysis software (Applied Cytometry Systems).

Inhibition of APC Antigen Processing

Autologous PBMC were irradiated, before being incubated for 10 min with 100 μM chloroquine in PBS at 37° C. Still in the presence of 100 μM chloroquine, the cells were then pulsed with LMP1 or IL-10-inducing LMP1 peptide for 3 h, before being washed three times. They were then used as a source of APC in proliferation and cytokine assays at 10⁶ cells/ml.

All documents cited herein are hereby incorporated by reference insofar as is required for the skilled person to carry out the invention.

### REFERENCES

Apostolopoulos V, Plebanski M. (2000) The evolution of DNA vaccines. Curr Opin Mol Ther 2(4):441-7
Baer, R., Bankier, A. T., Biggin, M. D., Deininger, P. L., Farrell, P. J., Gibson, T. J., Hatfull, G., Hudson, G. S., Satchwell, S. C., Seguin, C., et al. (1984). DNA sequence and expression of the B95-8 Epstein-Barr virus genome. Nature 310, 207-11.
Blaschke V, Reich K, Blaschke S, Zipprich S, Neumann C. (2000) Rapid quantitation of proinflammatory and chemoattractant cytokine expression in small tissue samples and monocyte-derived dendritic cells: validation of a new real-time RT-PCR technology. J Immunol Methods 246(1-2):79-90.
Chapman, A. L. N., Rickinson A. B., Thomas W. A., Jarrett R. F., Crocker J., and Lee S. P. (2001). Epstein-Barr virus-specific cytotoxic T lymphocyte responses in the blood and tumor site of Hodgkin's disease patients: implications for a T-cell-based therapy. Cancer Res. 61, 6219-26.
Christensen, J. P., Bartholdy, C., Wodarz, D., and Thomsen, A. R. (2001). Depletion of CD4+ T cells precipitates immunopathology in immunodeficient mice infected with a noncytocidal virus. J. Immunol. 166(5), 3384-91.
Demay F, Darche S, Kourilsky P, Delassus S. (1996) Quantitative PCR with colorimetric detection. Res Immunol 147(6):403-6.
Devereux, G., Hall, A. M., and Barker, R. N. (2000). Measurement of T-helper cytokines secreted by cord blood mononuclear cells in response to allergens. J Immunol Methods. 234, 13-22.
Dieckmann, D., Plottner, H., Berchtold, S., Berger, T., and Schuler, G. (2001). Ex vivo isolation and characterisation of CD4+ CD25+T cells with regulatory properties from human blood. J. Exp. Med. 193, 1303-1310.
Dukers, D. F., Meij, P., Vervoort, M. B., Vos, W., Scheper, R. J., Meijer, C. J., Bloemena, E., and Middeldorp, J. M. (2000). Direct immunosuppressive effects of EBV-encoded latent membrane protein 1. J Immunol. 165, 663-670.
Eliopoulos, A. G., Dawson, C. W., Mosialos, G., Floettmann, J. E., Rowe, M., Armitage, R. J., Dawson, J., Zapata, J. M., Kerr, D. J., Wakelam, M. J., Reed, J. C., Kieff, E., and Young, L. S. (1996). CD40-induced growth inhibition in epithelial cells is mimicked by Epstein-Barr Virus-encoded LMP1: involvement of TRAF3 as a common mediator. Oncogene. 13, 2243-2254.
Eliopoulos, A. G., Stack, M., Dawson, C. W., Kaye, K. M., Hodgkin, L., Sihota, S., Rowe, M., and Young, L. S. (1997). Epstein-Barr virus-encoded LMP1 and CD40 mediate IL-6 production in epithelial cells via an NF-kappaB pathway involving TNF receptor-associated factors. Oncogene. 14, 2899-2916.
Erickson, A. L., Kimura, Y., Igarashi, S., Eichelberger, J., Houghton, M., Sidney, J., McKinney, D., Sette, A., Hughes, A. L., and Walker, C. M. (2001). The outcome of Hepatitis C virus infection is predicted by escape mutation in epitopes targeted by cytotoxic T lymphocytes. Immunity 15, 883-895.
Fiorentino, D. F., Zlotnik, A., Vieira, P., Mosmann, T. R., Howard, M., Moore, K. W., O'Garra, A. (1991) IL-10 acts on the antigen-presenting cell to inhibit cytokine production by Th1 cells. J. Immunol. 146, 3444-3451.
Fries, K. L., Miller, W. E., and Raab-Traub, N. (1996). Epstein-Barr virus latent membrane protein 1 blocks p53-mediated apoptosis through the induction of the A20 gene. J. Virol. 70, 8653-8659.
Gorelik, L. and Flavell, R. A. (2000). Abrogation of TGF beta signalling in T cells leads to spontaneous T cell differentiation and autoimmune disease. Immunity 12, 171-181.
Gregory, C. D., Murray, R. J., Edwards, C. F., and Rickinson, A. B. (1988). Downregulation of cell adhesion molecules LFA-3 and ICAM-1 in Epstein-Barr virus-positive Burkitt's lymphoma underlies tumor cell escape from virus-specific T cell surveillance. J. Exp. Med. 167, 1811-1824.
Groux, H., O'Garra, A., Bigler, M., Rouleau, M., Antonenko, S., de Vries, J. E., and Roncarolo, M. G. (1997). A CD4+ T-cell subset inhibits antigen-specific T-cell responses and prevents colitis. Nature. 389 (6652), 737-742.
Haraguchi, S., Good, R. A., and Day, N. K. (1995) Immunosuppressive retroviral peptides: cAMP and cytokine patterns. Immunol. Today 16, 595.
Hayes, D. P., Brink, A. A., Vervoort, M. B., Middeldorp, J. M., Meijer, C. J., van den Brule, A. J (1999) Expression of Epstein-Barr virus (EBV) transcripts encoding homologues to important human proteins in diverse EBV associated diseases. Mol Pathol. 52, 97-103.
Horikawa, T., Yoshizaki, T., Sheen, T. S., Lee, S. Y., and Furukawa, M. (2000). Association of latent membrane protein 1 and matrix metalloproteinase 9 with metastasis in nasopharyngeal carcinoma. Cancer. 89, 715-23.
Huen, D. S., Henderson, S. A., Croom-Carter, D., and Rowe, M. (1995). The Epstein-Barr virus latent membrane protein-1 (LMP1) mediates activation of NF-kappa B and cell surface phenotype via two effector regions in its carboxy-terminal cytoplasmic domain. Oncogene. 10, 549-560.
Jonuleit, H., Schmitt, E., Stassen, M., Tuettenberg, A., Knop, J., and Enk, A. (2001). Identification and Functional Characterization of Human CD4+ CD25+T Cells with Regulatory Properties Isolated from Peripheral Blood. J. Exp. Med. 193, 1285-1294.
Kamel-Reid S, Dick J E. Engraftment of immune-deficient mice with human hematopoietic stem cells. Science 1988; 242:1706-9.
Khanna, R., Burrows, S. R., Nicholls, J., and Poulsen, L. M. (1998). Identification of cytotoxic T cell epitopes within Epstein-Barr virus (EBV) oncogene latent membrane protein 1 (LMP1): evidence for HLA A2 supertype-restricted immune recognition of EBV-infected cells by LMP1-specific cytotoxic T lymphocytes. Eur J. Immunol. 28, 451-458.
Kieff, E. (1996) Epstein-Barr virus and its replication. In Fields Virology, Fields B. N., Knipe, D. M. and Howley, P. M eds. (Lipincott-Raven Publishers, Philadelphia), pp2343-2396.
Kjellstrom S, Emneus J, Marko-Varga G. (2000) Flow immunochemical bio-recognition detection for the determination of interleukin-10 in cell samples. J Immunol Methods 246(1-2):119-30.
Kreft B, Singer G G, Diaz-Galloii C, Kelley V R. (1992) Detection of intracellular interleukin-10 by flow cytometry. J Immunol Methods 156(1):125-8
Levings, M. K. and Roncarolo, M. G. (2000). T-regulatory 1 cells: a novel subset of CD4 T cells with immunoregulatory properties. J Allergy Clin Immunol. 106, S109-112.
Levings, M. K., Sangregorio, R., and Roncarolo, M. G. (2001). Human CD25+CD4+ T regulatory cells suppress naïve and memory T cell proliferation and can be expanded in vitro without loss of function. J. Exp. Med. 193, 1295-1302.
Levitskaya, J., Coram, M., Levitsky, V., Imreh, S., Steigerwald-Mullen, P. M., Klien, G., Kurilla, M. G., and Masucci, M. G. (1995). Inhibition of antigen processing by the internal repeat region of the Epstein-Barr virus nuclear antigen-1. Nature 375, 685-688.
Levitskaya, J., Sharipo, A., Leonchiks, A., Ciechanover, A., and Masucci, M. G. (1997). Inhibition of ubiquitin/proteasome-dependent protein degradation by the Gly-Ala repeat domain of the Epstein-Barr virus nuclear antigen 1. Proc. Natl. Acad. Sci. USA 94, 12616-12621.
Lorenzo, M. E., Ploegh, H. L., and Tirabassi, R. S. (2001). Viral immune evasion strategies and the underlying cell biology. Semin Immunol. 13, 1-9.
MacDonald, A J, Duffy, M, Brady, M T, McKiernan, S, Hall, W, Hegarty, J, Curry, M, and Mills K H G. CD4 T Helper Type 1 and Regulatory T Cells Induced against the Same Epitopes on the Core Protein in Hepatitis C Virus-Infected Persons. The Journal of Infectious Diseases (2002) 185:720-7
McCune J M, Namikawa R, Kaneshima H, Shultz L D, Lieberman M, Weissman I L. The SCID-hu mouse: murine model for the analysis of human hematolymphoid differentiation and function. Science 1988; 241:1632-9.
McGuirk, P., C. McCann, and K. H. G. Mills. 2002. Pathogen-specific T regulatory 1 cells induced in the respiratory tract by a bacterial molecule that stimulates interleukin 10 production by dendritic cells: a novel strategy for evasion of protective T helper type 1 responses by Bordetella pertussis. J. Exp. Med. 195: 221.
Mills, K. H. G., O. Leavy, P. McGuirk, S. Higgins, E. McNeela, C. McCann, M. E. Armstrong, and E. Lavelle. 2002. Pathogen-derived immunomodulatory molecules: the bridge between innate and adaptive immunity. Immunology 107(Suppl. 1):8.
Mor G, Eliza M. (2001) Plasmid DNA vaccines. Immunology, tolerance, and autoimmunity. Mol Biotechnol 19(3):245-50
Mosialos, G., Birkenbach, M., Yalamanchili, R., VanArsdale, T., Ware, C., and Kieff, E. (1995). The Epstein-Barr virus transforming protein LMP1 engages signaling proteins for the tumor necrosis factor receptor family. Cell. 80, 389-399.
Mosier D E, Gulizia R J, Baird S M, Wilson D B. Transfer of a functional human immune system to mice with severe combined immunodeficiency. Nature 1988 335:256-9
Mossman, T. R., and Coffman, R. L. (1989). Heterogeneity of cytokine secretion patterns and functions of helper T-cells. Adv. Immunol. 46, 111-147.
Mueller, Y. M., De Rosa, S. C., Hutton, J. A., Witek, J., Roederer, M., Altman, J. D., and Katsikis, P. D. (2001). Increased CD95/Fas-Induced apoptosis of HIV-Specific CD8+ T cells. Immunity 15, 883-895.
Pallesen, G., Hamilton-Dutoit, S. J., Rowe, M. and Young, L. S. (1991) Expression of Epstein-Barr virus latent gene products in tumour cells of Hodgkin's disease. Lancet. 337, 320-322.
Plebanski, M., Saunders, M., Burtles, S. S., Crowe, S. and Hooper, D. C. (1992). Primary and secondary human in vitro T-cell responses to soluble antigens are mediated by subsets bearing different CD45 isoforms. Immunology. 75, 86-91.
Roncarolo, M. and Levings, M. K. (2000). The role of different subsets of T regulatory cells in controlling autoimmunity. Curr Opin Immunol. 12, 676-683.
Roncarolo, M. G., Levings, M. K., and Traversari, C. (2001). Differentiation of T Regulatory Cells by Immature Dendritic cells. J. Exp. Med. 193, F5-F9.
Rowe M, Young L S, Crocker J, Stokes H, Henderson S, Rickinson A B. Epstein-Barr virus (EBV)-associated lymphoproliferative disease in the SCID mouse model: implications for the pathogenesis of EBV-positive lymphomas in man. J Exp Med 1991; 173:147-58.
Scheffold A, Assenmacher M, Reiners-Schramm L, Lauster R, Radbruch A. (2000) High-sensitivity immunofluorescence for detection of the pro- and anti-inflammatory cytokines gamma interferon and interleukin-10 on the surface of cytokine-secreting cells. Nat Med 6(1):107-10.
Schlaak J F, Schmitt E, Huls C, Meyer zum Buschenfelde K H, Fleischer B. (1994) A sensitive and specific bioassay for the detection of human interleukin-10. J Immunol Methods 168(1):49-54.
Schleef M, Schmidt T, Flaschel E. (2000) Plasmid DNA for pharmaceutical applications. Dev Biol (Basel) 104:25-31
Seddon, B., and Mason, D. (2000). The third function of the thymus. Immunol. Today 21, 95-99.
Shevach, E. M. (2000). Regulatory T cells in autoimmmunity. Ann. Rev. Immunol. 18, 423-449.
Shevach, E. M., Thornton A., Suri-Payer E., (1998). T lymphocyte-mediated control of autoimmunity. Novartis Found Symp. 215, 200-211.
Singh, H. and Raghava, G. P. S. (2001) ProPred: Prediction of HLA-DR binding sites. Bioinformatics, 17(12), 1236-37.
Smith H A. (2000) Regulation and review of DNA vaccine products. Dev Biol (Basel) 104:57-62.
Spencer, J. V., Lockridge, K. M., Barry, P. A., Lin, G., Tsang, M., Penfold, M. E. T., and Schall, T. J. (2002). Potent immunosuppressive activities of Cytomegalovirus encoded Interleukin-10. J. Virol. 76, 1285-1292.
Stephens, L. A., and Mason, D. (2000). CD25 is a marker for CD4+ thymocytes that prevent autoimmune diabetes in rats, but peripheral T cells with this function are found in both CD25+ and CD25− subpopulations. J. Immunol. 165, 3105-3110.
Stott, L. M., Barker, R. N., and Urbaniak, S. J. (2000). Identification of alloreactive T-cell epitopes on the Rhesus D protein. Blood. 96, 4011-4019.
Sturniolo. T., Bono. E., Ding. J., Raddrizzani. L., Tuereci. O., Sahin. U., Braxenthaler. M., Gallazzi. F., Protti. M. P., Sinigaglia. F., Hammer. J., Generation of tissue-specific and promiscuous HLA ligand database using DNA microarrays and virtual HLA class II matrices. Nat. Biotechnol. 17. 555-561(1999).
Taylor, P. A., Noelle, R. J., and Blazar, B. R. (2001). CD4+ CD25+ immune regulatory cells are required for induction of tolerance to alloantigen via costimulatory blockade. J. Exp. Med. 193, 1311-1318.
Thomsen, A. R., (2001). The outcome of viral infection is determined by an interplay between CD4+ and CD8+ T cells. Immunology 104, (suppl. 1) 40.
Urban, B. C., N. Willcox, and D. J. Roberts. 2001. A role for CD36 in the regulation of dendritic cell function. Proc. Natl. Acad. Sci. USA 98:8750.
Weiner, H. L. (1997). Oral tolerance: Immune mechanisms and treatment of autoimmune diseases. Immunol. Today 18, 335-343.
Xu, X.-N., Screatom, G. R., and McMichael, A. J. (2001). Virus Infections: Escape, Resistance, and Counterattack. Immunity 15, 867-870.
Young, L. S., Eliopoulos, A. G., Gallagher, N. J., and Dawson, C. W. (1998). CD40 and epithelial cells: across the great divide. Immunol Today. 19, 502-506.

1. A method of tolerising a cell population to a target antigen, comprising contacting said cell population with
(a) a tolerogenic peptide sequence from EBV LMP1 or LMP2 protein, or a nucleic acid encoding said tolerogenic peptide sequence such that said tolerogenic peptide sequence is expressed in said cell population; and
(b) the target antigen, or a nucleic acid encoding said target antigen such that said target antigen is expressed in said cell population; wherein said cell population comprises mononuclear leukocytes from a subject seropositive for EBV.

2. A method according to claim 1, comprising the steps of contacting a population of antigen presenting cells with said tolerogenic peptide sequence and said target antigen, and subsequently contacting said cell population with said population of antigen presenting cells.

3. A method according to claim 1, optionally comprising the steps of contacting a population of antigen presenting cells with said tolerogenic peptide sequence and said target antigen, and subsequently contacting said cell population with said population of antigen presenting cells wherein said mononuclear leukocytes are contacted with said tolerogenic peptide sequence and said target antigen in vitro.

4. A method according to claim 3, wherein said cell population or a subset thereof is re-administered to said subject after contacting with said tolerogenic peptide sequence and said target antigen.

5. A method according to claim 2, wherein said population of antigen presenting cells is contacted with said tolerogenic peptide sequence and said target antigen in vitro and said cell population is contacted with said population of antigen presenting cells in vivo.

6. A method according to claim 1, wherein said tolerogenic peptide sequence and said target antigen are administered directly to said subject.

7. A method according to claims 1, wherein the tolerogenic peptide sequence comprises one or more of the sequences P2, P4, P7, P14, PIS, PIS, P20, P22, 1323, P24 and P32.

8. A pharmaceutical composition for tolerisation of an individual against a target antigen, said composition comprising a molecule selected from the group consisting of EBV LMP1, LMP2, a tolerogenic peptide sequence thereof, or a nucleic acid encoding the same wherein the composition further comprises the target antigen or a nucleic acid encoding the target antigen in a pharmaceutically acceptable carrier, and wherein the individual has previously been infected with EBV.

9. The composition according to claim 8, wherein the composition comprises the target antigen or a nucleic acid encoding the target antigen.

10. The composition according to claim 8 wherein the target antigen is a cell.

11. The composition according to claim 10 wherein the cell is for transplantation.

12. The composition according to claim 11 wherein the cell comprises nucleic acid encoding the tolerogenic peptide sequence.

13. The composition according to claim 8 wherein the tolerogenic peptide sequence comprises one or more of the sequences P2, P4, P7, P14, P15, P18, P20, P22, P23, P24 and P32.

14. (canceled)

15. A method for assessing the tolerogenicity of a test peptide sequence from an infectious agent, comprising the steps of:
(i) contacting a cell population with said test peptide sequence,
(ii) determining whether IL-10 expression in said cell population is increased, and optionally
(iii) correlating the result of step (ii) with the tolerogenicity of the sequence,
wherein said infectious agent is optionally a virus and said cell population comprises mononuclear leukocytes from a donor previously infected by said infectious agent.

16. A method according to claim 15, wherein said cell population comprises at least one type of antigen presenting cell.

17. A method according to claim 15, wherein said cell population optionally comprises at least one type of antigen presenting cell and said mononuclear leukocytes comprise at least one cell type selected from the group consisting of T lymphocytes, B lymphocytes, natural killer (NK) cells, monocytes, macrophages or dendritic calls.

18. A method according to claim 17, wherein said mononuclear leukocytes comprise at least CD4⁺ T lymphocytes.

19. A method according to claim 18, wherein said mononuclear leukocytes further comprise at least one type of antigen presenting cell.

20. A method according to claims 15, further comprising the steps of:
(i) (a) contacting a similar cell population from a donor not previously infected by said infectious agent with said test peptide sequence, said cell population optionally comprising at least one type of antigen presenting cell and said infectious agent optionally being a virus; and
(ii)(a) determining whether IL-10 expression in said cell population is increased, and optionally
(ii) (b) comparing the results from step (ij) with the results from step (ii)(a).

21. A method according to claims 15, wherein the infectious agent is a virus.

22. A method according to claim 21, wherein the virus is a herpesvirus encoding a viral IL-10 homologue.

23. A method according to claim 22, wherein the virus is EBV.

24. A method according to claim 23, wherein the test peptide sequence is derived from EBV LMP1 protein or LMP2 protein.

25. A method according to claim 24, wherein the test or tolerogenic peptide sequence comprises one or more of the sequences P1 to P75 or P1′ to P96′.

26. A method for assessing the tolerogenicity of a test peptide sequence from an infectious agent towards a target antigen, comprising the steps of:
(i) contacting a cell population with (a) said test peptide sequence and (b) a target antigen, to make a test composition,
(ii) re-contacting the cell population from said test composition with said target antigen in the absence of said test peptide sequence,
(iii) assessing cell proliferation or expression of IL-4, IL-2, IL-12 or gamma-IFN by said cell population in response to said target antigen, and optionally
(iv) correlating the result of step (iii) with the tolerogenicity of the test peptide sequence, wherein said cell population comprises mononuclear leukocytes from a donor previously infected by said infectious agent.

27. A method according to claim 26, further comprising the step of adding fresh antigen presenting cells prior to step (ii).

28. A method according to claim 26 optionally comprising the step of adding fresh antigen presenting cells prior to step (ii), said method further comprising the step of contacting the cell population with a confirmatory antigen unrelated to the test sequence or the target antigen.

29. A method according to claim 26, wherein the infectious agent is a virus.

30. A method according to claim 29, wherein the virus is a herpesvirus encoding a viral IL-10 homologue.

31. A method according to claim 30, wherein the virus is EBV.

32. A method according to claim 31, wherein the test peptide sequence is derived from EBV LMP1 protein or LMP2 protein.

33. A method according to claim 32, wherein the test or tolerogenic peptide sequence comprises one or more of the sequences P1 to P75 or P1′ to P96′.

34. A method for assessing the tolerogenicity of a test peptide sequence, comprising the steps of:
(i) contacting a first cell population with said test peptide sequence,
(ii) contacting a second cell population with a control peptide sequence
(iii) determining whether IL-10 expression in each said cell population is increased and optionally
iv) correlating the result of step (iii) with the tolerogenicity of the test peptide sequence, wherein each said cell population comprises mononuclear leukocytes from a donor previously infected by an infectious agent, and said control peptide sequence is derived from said infectious agent.

35. A method according to claim 34 wherein said control peptide sequence has previously been identified to induce IL-10 expression in a cell population comprising mononuclear leukocytes from a donor previously infected by said infectious agent, said infectious agent optionally being EBV.

36. A method according to claim 34, wherein said control peptide sequence has previously been identified to induce IL-10 expression in a cell population comprising mononuclear leukocytes from a donor previously infected by said infectious agent and wherein said first and second cell populations are derived from the same donor, said infectious agent optionally being EBV.

37. A method according to claim 36, wherein said first and second cell populations comprise a T cell clone capable of proliferating in response to the control peptide.

38. A method according to claim 34, wherein said infectious agent is EBV.

39. A method according to claim 34, wherein said control peptide is derived from LMP1 or LMP2.

40. A peptide having the sequence of any one of P2, P4, P5, P6, P7, P8, P9, P10, P12, p13, p14, p15, p16, P17, P18, P20, P22, P23, P24, P25, P26, P27, P29, P30, P32, P34, P35, P39, P68, P71, and P72.
